(19) United States
(12) Patent Application Publication
(10) Pub. No.: US 2024/0316054 A1
(43) Pub. Date: Sep. 26, 2024

(54) NOVEL PLK1 PROTEIN DEGRADATION-INDUCING COMPOUND

(71) Applicant: UPPTHERA, INC., Incheon (KR)

(72) Inventors: Hye Guk Ryu, Incheon (KR); Keum Young Kang, Incheon (KR); Im Suk Min, Gyeonggi-do (KR); Seong Hoon Kim, Incheon (KR)

(73) Assignee: UPPTHERA, INC., Incheon (KR)

(21) Appl. No.: 18/574,867

(22) PCT Filed: Jun. 29, 2022
(86) PCT No.: PCT/KR2022/009343

§ 371 Date: Dec. 28, 2023

(30) Foreign Application Priority Data
Jun. 30, 2021 (KR) 10-2021-0085980

(51) Int. Cl. A61K 31/519; C07D 487/04

(52) U.S. Cl. CPC A61K 31/519; C07D 487/04

(57) ABSTRACT

The present invention relates to a PLK1 protein degradation inducing compound, a preparation method thereof, and a use thereof. Compounds of the present invention target PLK1 and BRD4 to degrade PLK1, and decompose BRD4 to a suitable level within the scope of causing no side effects, and thus can be very advantageously used for prevention or treatment of cancer or neural disease.

### TECHNICAL FIELD

The present invention relates to a PLK1 protein degradation inducing compound, a method for preparing thereof, and the use thereof.

### BACKGROUND ART

Polo-like kinase 1 (PLK1) is a serine/threonine kinase and is known to be involved in the G2/M phase transition during cell growth and division. PLK1 is expressed and activated in a pulse form from S phase to G2/M phase, and rapidly degrades as mitosis ends. PLK1 is overexpressed in various cancer types, including colorectal cancer, lung cancer, bladder cancer, melanoma, and the like, and cancer cells overexpressing PLK1 tend to show resistance to various types of anticancer drugs. As the PLK1 dependence in various cancer types was revealed as described above, there have been attempts to develop PLK1 inhibitor compounds such as volasertib (also known as BI6727), etc.

However, the conventional PLK1 inhibitors failed to sufficiently inhibit PLK1 activity at concentrations that are clinically safe. In other words, there is a problem that even if the cell cycle of cancer cells is temporarily delayed, the cell cycle eventually restarts in some cancer cells, which may not obtain sufficient clinical effects (see Gheghiani et al., Cell Reports, 2017, 19: 2060, etc.). In fact, a number of pharmaceutical companies, including Boehringer Ingelheim, GlaxoSmithKline, etc., have attempted to develop small-molecular compound-based PLK1 inhibitors, but most of them have failed or stopped in the clinical trial stage, and thus there are no commercially available PLK1 inhibitors to date. This suggests that mechanisms of inhibiting enzyme activity by binding to the active site of PLK1, such as small molecule inhibitors, are not sufficiently effective. Therefore, there is a need to develop new methods of PLK1-targeted treatments rather than small molecule compounds.

Meanwhile, proteolysis targeting chimera (PROTAC) has recently been proposed as a small molecule-based platform technology capable of inducing proteolysis of target proteins in the body. The PROTAC is a bifunctional compound in which a ligand molecule that binds to disease-related target protein and an E3 ubiquitin ligase binding moiety are linked by a chemical linker. The PROTAC compound is capable of inducing degradation of the target protein by placing the disease-related target protein near the E3 ubiquitin ligase. In the case of the PROTAC compound using PLK1 as a target protein, Chinese Patent Laid-Open No. 106543185 discloses some PROTAC compounds in which a volasertib derivative compound and a binding moiety for the E3 ubiquitin ligase CRBN are linked by a chemical linker. The PROTAC compound is a compound that is characterized by simultaneously degrading PLK1 and BRD4 (bromodomain 4). Like PLK1 described above, BRD4 is also known to be a high potential therapeutic target in various cancer types. BRD4 is a protein belonging to the bromodomain and extraterminal domain (BET) family and has been reported to be effective in midline carcinoma, acute myeloid leukemia, multiple myeloma, pancreatic cancer, and the like, by regulating the expression of key proto-oncogenes such as c-Myc, Bcl-xL, and Bcl-2, and inhibiting BRD4. In addition, it has recently been reported that inhibition of PLK1 and BRD4 has a synergistic effect in patients with acute myeloid leukemia and pancreatic cancer (Mu et al., Biochem Biophys Res Commun., 2020, 521(4): 833), and thus expectations are rising for the development of PROTAC capable of simultaneously degrading PLK1 and BRD4.

However, it has been reported that excessively strong inhibition of BRD4 activity is inevitably accompanied by on-target side effects such as blood toxicity and gastrointestinal toxicity together with pharmacological effects (Bolden et al., Cell Reports, 2014, 8 (6): 1919). Therefore, when developing PROTACs that simultaneously target PLK1 and BRD4, it is very important to appropriately control the level of degradation of BRD4 to minimize the possibility of side effects.

### DISCLOSURE

### Technical Problem

The present inventors made a diligent effort to develop a novel proteolysis targeting chimera (PROTAC) compound that targets PLK1 and BRD4 simultaneously without exhibiting toxicity, and as a result, completed the present invention by discovering the novel PROTAC compound that has strong degradation activity against PLK1 while degrading BRD4 to an appropriate level.

### Technical Solution

An object of the present invention is to provide a polo-like kinase 1 (PLK1) protein degradation inducing compound.

Another object of the present invention is to provide a pharmaceutical composition for preventing or treating cancer or neurological diseases, comprising the PLK1 protein degradation inducing compound as an active ingredient.

### Advantageous Effects

The compounds according to the present invention may target PLK1 and BRD4 to degrade PLK1 and also degrade BRD4 to an appropriate level of causing no side effects, and thus can be very advantageously used for prevention or treatment of cancer or neurological diseases.

### DESCRIPTION OF THE DRAWINGS

FIG. 1 shows the degradation activity of the compounds of the present invention against PLK1 and BRD4.

### BEST MODE FOR CARRYING OUT THE INVENTION

The present disclosure will be described in detail as follows. Meanwhile, each description and embodiment disclosed in the present disclosure may be applied to each of the other descriptions and embodiments. In other words, all combinations of various elements disclosed in the present disclosure fall within the scope of the present disclosure. In addition, it cannot be considered that the scope of the present disclosure is limited by specific descriptions described below.

One aspect of the present invention for achieving the above object is a compound represented by the following Chemical Formula I, a stereoisomer thereof, or a pharmaceutically acceptable salt thereof:

### [Chemical Formula I]

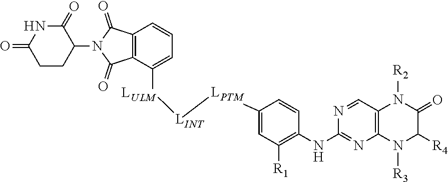 [0014] in Chemical Formula I above,
R₁ is —H, —CH₃ or —OCH₃,
R₂ is —H or —CH₃,
R₃ is -cyclopentyl or -cyclohexyl,
R₄ is —C₁₋₃alkyl,
L_{ULM} is —NH—CH₂CH₂— or

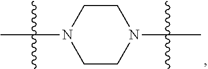 [SMILES: *N1CCN(*)CC1]

L_{INT} is —(OCH₂CH₂)ₙ—, —(C═O)—CH₂CH₂—(C═O)—, —CH₂CH₂—(C═O)—, —CH₂—(C═O)—,

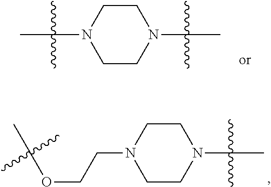

wherein n is an integer of 2 to 4, and L_{PTM} is —NH—(C═O)—,

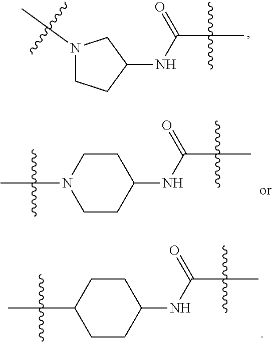 [SMILES: *C(=O)NC1CCN(*)C1 ; *C(=O)NC1CCN(*)CC1 ; *C(=O)NC1CCC(*)CC1]

The compound represented by Chemical Formula I is a bifunctional compound in which a target protein binding moiety and an E3 ubiquitin ligase binding moiety are linked by a chemical linker. In Chemical Formula I, the linker is represented by -L_{ULM}-L_{INT}-L_{PTM}-. The compound represented by Chemical Formula I of the present invention has the activity of targeting and degrading PLK1 and BRD4, and furthermore, degrades BRD4 to an appropriate level rather than completely degrading BRD4, thereby having anti-cancer activity while simultaneously reducing the risk of side effects. In previous studies, BRD4 knockdown mice were observed to have epidermal hyperplasia, hair loss, decreased cell diversity, and a decrease in stem cells in the small intestine, and it was reported that intestinal regeneration ability was impaired after radiation (Bolden et al., Cell Reports, 2014, 8(6): 1919). Therefore, excessive inhibition of BRD4 is expected to have a significant negative effect on various organs, and the compounds of the present invention, which exhibit an appropriate level of inhibitory activity against BRD4, have a clinically advantageous effect in that side effects are reduced.

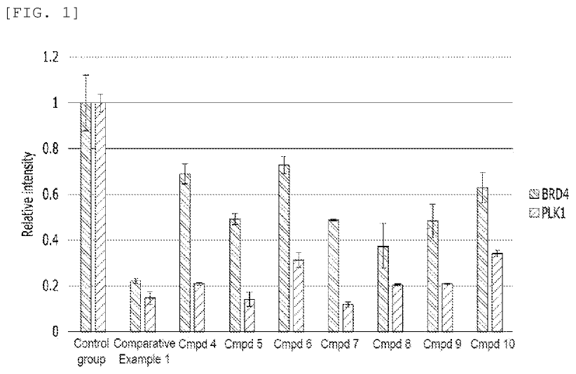

In a specific example of the present invention, it was confirmed that by treating cells stably expressing PLK1 or BRD4 with the compounds of the present invention, the PLK1 protein was mostly degraded and the BRD4 protein was degraded at a relatively weak level (FIG. 1 and Table 2). Therefore, the compound represented by Chemical Formula I of the present invention may exhibit a strong level of inhibition against PLK1 and a weak level of inhibition against BRD4, to be usable for the prevention or treatment of cancer or neurological diseases without side effects compared to conventionally known PROTAC substances.

In the present invention, the compound represented by Chemical Formula I may be Compound 1 to Compound 11 shown in Table 1 below.

TABLE 1

| Com- | |
|---|---|
| pound | Structure |
| 1 | 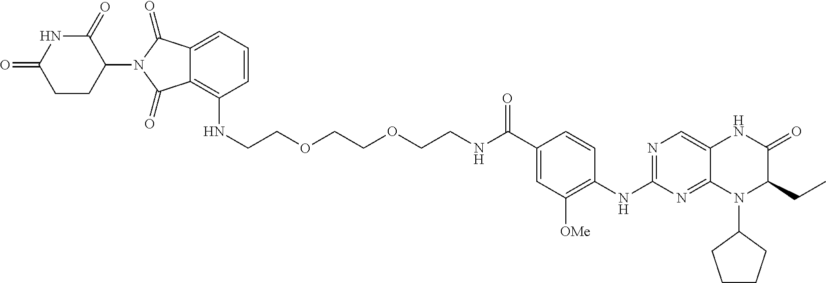 [SMILES: CC[C@@H]1C(=O)Nc2cnc(Nc3ccc(C(=O)NCCOCCOCCNc4cccc5c4C(=O)N(C4CCC(=O)NC4=O)C5=O)cc3OC)nc2N1C1CCCC1] |
| 2 | 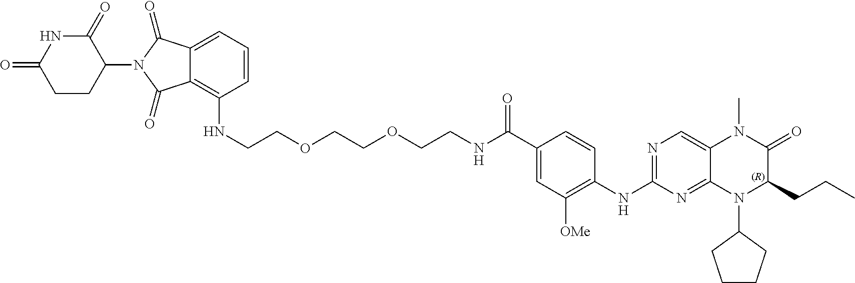 |
| 3 | 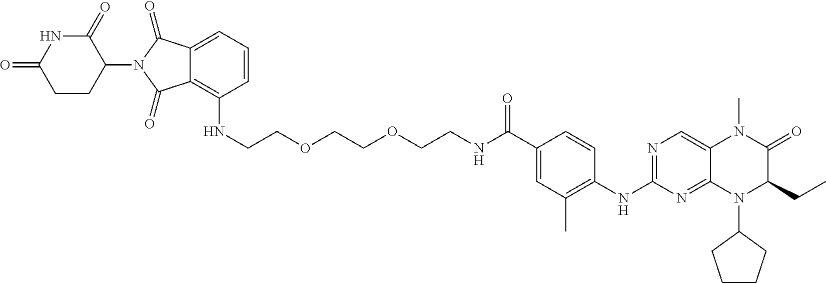 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3C)nc2N1C1CCCC1] |
| 4 | 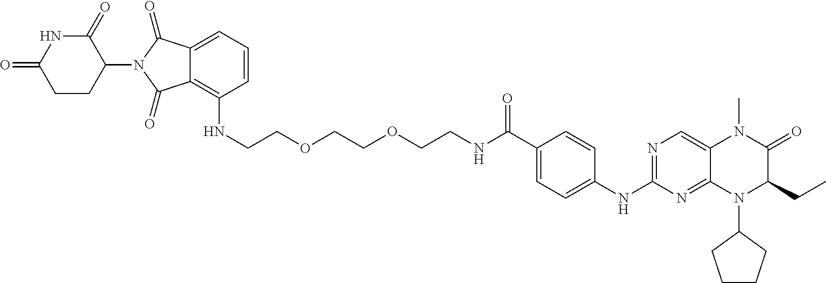 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3)nc2N1C1CCCC1] |
| 5 | 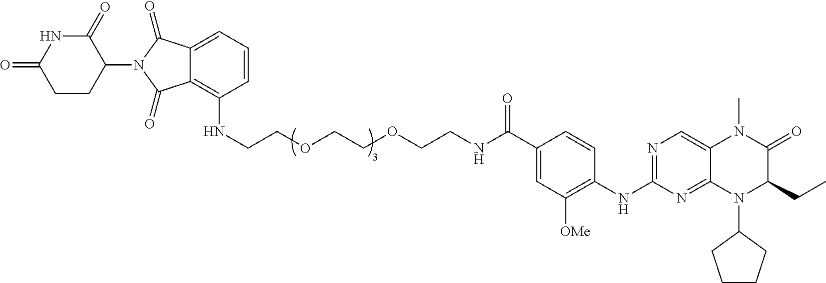 |
| 6 | 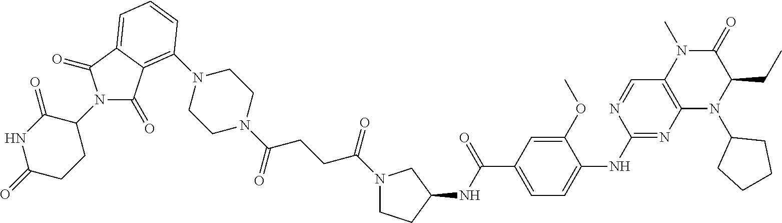 [SMILES: CC[C@@H]1C(=O)N(C)c2cnc(Nc3ccc(C(=O)N[C@H]4CCN(C(=O)CCC(=O)N5CCN(c6cccc7c6C(=O)N(C6CCC(=O)NC6=O)C7=O)CC5)C4)cc3OC)nc2N1C1CCCC1] |
| 7 | 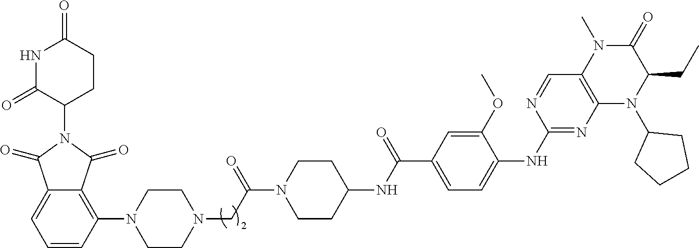 |
| 8 | 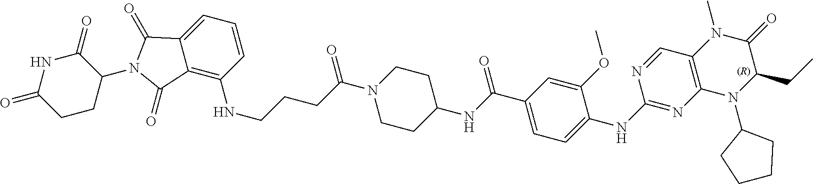 |
| 9 | 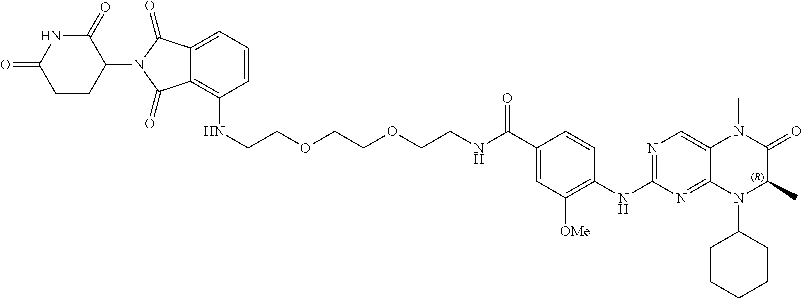 |
| 10 | 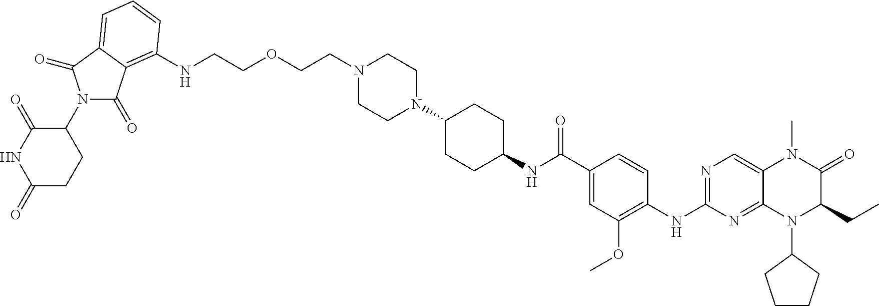 [SMILES: CC[C@@H]1C(=O)N(C)c2cnc(Nc3ccc(C(=O)N[C@H]4CC[C@H](N5CCN(CCOCCNc6cccc7c6C(=O)N(C6CCC(=O)NC6=O)C7=O)CC5)CC4)cc3OC)nc2N1C1CCCC1] |
| 11 | 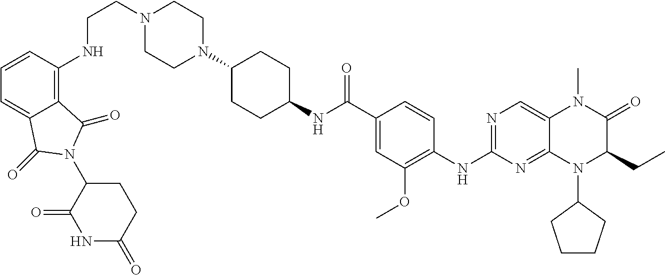 [SMILES: CC[C@@H]1C(=O)N(C)c2cnc(Nc3ccc(C(=O)N[C@H]4CC[C@H](N5CCN(CCNc6cccc7c6C(=O)N(C6CCC(=O)NC6=O)C7=O)CC5)CC4)cc3OC)nc2N1C1CCCC1] |

As used herein, the term “pharmaceutically acceptable salt” is used to refer to an acid addition salt or base addition salt that is suitable or compatible for the treatment of patients. Exemplary inorganic acids that form suitable salts may include not only hydrochloric acid, hydrobromic acid, sulfuric acid, and phosphoric acid, but also metal salts such as sodium monohydrogen orthophosphate and potassium hydrogen sulfate. Exemplary organic acids that form suitable salts may include not only mono-, di- and tricarboxylic acids such as glycolic acid, lactic acid, pyruvic acid, malonic acid, succinic acid, glutaric acid, fumaric acid, malic acid, tartaric acid, citric acid, ascorbic acid, maleic acid, benzoic acid, phenylacetic acid, cinnamic acid, and salicylic acid, but also sulfonic acids such as p-toluenesulfonic acid and methanesulfonic acid. Monoacid or diacid salts may be formed, and these salts may be present in hydrated, solvated or substantially anhydrous form. In general, the acid addition salts of the compounds of the present invention are more soluble in water and various hydrophilic organic solvents, and generally exhibit higher melting points, as compared to free base forms thereof. Selection of appropriate salts is known to those skilled in the art. Other non-pharmaceutically acceptable salts, such as oxalates, may be used in the isolation of the compounds of the present invention, for example, for laboratory use or for subsequent conversion to pharmaceutically acceptable acid addition salts. Exemplary inorganic bases that form suitable salts may include lithium, sodium, potassium, calcium, magnesium, or barium hydroxide. Exemplary organic bases that form suitable salts may include aliphatic, cycloaliphatic or aromatic organic amines such as methylamine, trimethylamine and picoline or ammonia. Accordingly, in some instances, salts contemplated by the present invention may include alkyl, dialkyl, trialkyl, or tetra-alkyl ammonium salts. In certain embodiments, salts contemplated by the present invention may include, but are not limited to, L-arginine, benentamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts.

Further, the compounds of the present invention include, without limitation, all possible optical isomers as well as pharmaceutically acceptable salts thereof. Stereoisomers of the compounds of the present invention may be prepared using methods known in the art.

In addition, the compounds of the present invention may be prepared in crystalline or amorphous form. Once the compound is prepared in crystalline form, the compound may optionally be hydrated or solvated.

The compounds of the present invention may be formulated as any therapeutic pharmaceutical composition or in a form suitable for use in any of the methods disclosed herein, such as preventive or therapeutic methods.

Another aspect of the present invention for achieving the above object is a pharmaceutical composition for preventing or treating cancer or neurological diseases, comprising the compound represented by the Chemical Formula I above, a stereoisomer thereof, or a pharmaceutically acceptable salt thereof as an active ingredient.

Still another aspect of the present is a method for preventing or treating cancer or neurological diseases, comprising administering the compound represented by the Chemical Formula I above, a stereoisomer thereof, or a pharmaceutically acceptable salt thereof to a subject in need thereof.

Still another aspect of the present invention is the use of the compound represented by Chemical Formula I, a stereoisomer thereof, or a pharmaceutically acceptable salt thereof for preventing or treating cancer or neurological diseases.

The compound represented by Chemical Formula I, a stereoisomer thereof, or a pharmaceutically acceptable salt thereof are the same as described above.

The compounds of the present invention are capable of fundamentally degrading the target protein PLK1, and thus it is possible to achieve an excellent PLK1 inhibitory effect and show excellent pharmacological effects due to PLK1 inhibition compared to conventional PLK1 small molecule inhibitors that simply inhibit the PLK1 activity. In addition, the compounds of the present invention may also degrade BRD4, another target protein, to an appropriate level, thereby making it possible to achieve the desired pharmacological effect and minimize side effects caused by excessive inhibition of BRD4. Therefore, the pharmaceutical composition comprising the compound represented by Chemical Formula I of the present invention, a stereoisomer thereof or a pharmaceutically acceptable salt thereof is very useful for the prevention or treatment of cancer or neurological diseases by inhibiting PLK1 and BRD4.

As used herein, the term “subject” or “patient” includes human and non-human animals. Non-human animals include vertebrates, such as mammals and non-mammals, including non-human primates, sheep, cats, horses, cows, chickens, dogs, mice, rats, goats, rabbits, and pigs. Preferably, the subject is a human. Except where noted, the terms “patient” or “subject” are used interchangeably herein.

As used herein, the terms “treat,” “treating,” or “treatment” of any disease, condition, or disorder include alleviating or improving a disease, condition, or disorder (i.e., delaying or preventing the occurrence of at least one of the condition or clinical symptoms thereof); or alleviating or improving at least one physical parameter or biomarker associated with a disease, condition or disorder, including those that are not perceptible to the patient.

As used herein, the terms “prevent,” “preventing,” or “prevention” of any disease, condition or disorder include preventive treatment of a disease, condition or disorder; or delaying the onset or progression of a condition or disorder.

In the present invention, cancer includes all cancers capable of exhibiting preventive or therapeutic efficacy due to inhibition of the activity of PLK1 and BRD4, and may be a solid cancer or a hematological cancer. For example, the cancer may be at least one selected from the group consisting of squamous cell carcinoma, small cell lung cancer, non-small cell lung cancer, adenocarcinoma of the lung, squamous cell carcinoma of the lung, peritoneal cancer, skin cancer, skin or intraocular melanoma, rectal cancer, perianal cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular cancer, gastrointestinal cancer, stomach cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulva cancer, thyroid cancer, head and neck cancer, brain cancer, osteosarcoma, and the like, but is not limited thereto. The cancer includes metastatic cancer as well as primary cancer.

In the present invention, the neurological diseases include all neurological diseases capable of exhibiting preventive or therapeutic efficacy due to inhibition of the activity of PLK1 and BRD4, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig's disease, stroke, and nerve damage and axonal degeneration-related disorders following brain or spinal cord injury, but is not limited thereto.

As used herein, the “composition” or “pharmaceutical composition” may further include one or more pharmaceutically acceptable carriers in addition to the compound of the present invention to facilitate administration of the compound to a patient or subject, but is not limited thereto. The pharmaceutically acceptable carrier is a non-active ingredient, that is, a pharmaceutically acceptable excipient, and may be appropriately selected by a person skilled in the art by referring to the document [see Handbook of Pharmaceutical Excipients], etc. Suitable routes of administration include, but are not limited to, oral administration, parenteral administration, such as intramuscular, intravenous, subcutaneous administration, etc.

The pharmaceutical composition of the present invention may further comprise one or more active ingredients exhibiting the same or similar efficacy in addition to the compound represented by Chemical Formula I, a stereoisomer thereof or a pharmaceutically acceptable salt thereof.

Exemplary embodiments of the present disclosure may be modified in various other forms, and the scope of the present disclosure is not limited to the exemplary embodiments to be described below. In addition, the exemplary embodiments of the present disclosure are provided to more completely explain the present disclosure to an ordinary person skilled in the art. Further, “including” a component throughout the specification does not mean excluding other components, but rather it means that other components may be further included, unless otherwise stated.

### MODE FOR CARRYING OUT THE INVENTION

Hereinafter, the constitution and effects of the present invention will be described in more detail through the following Examples. These Examples are only provided for illustrating the present invention, but the scope of the present invention is not limited by these Examples.

### Example 1: Synthesis of 4-(((R)-8-cyclopentyl-7-ethyl-6-oxo-5, 6, 7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methoxybenzamide (Compound 1)

### Synthetic Scheme:

### Experimental Procedure:

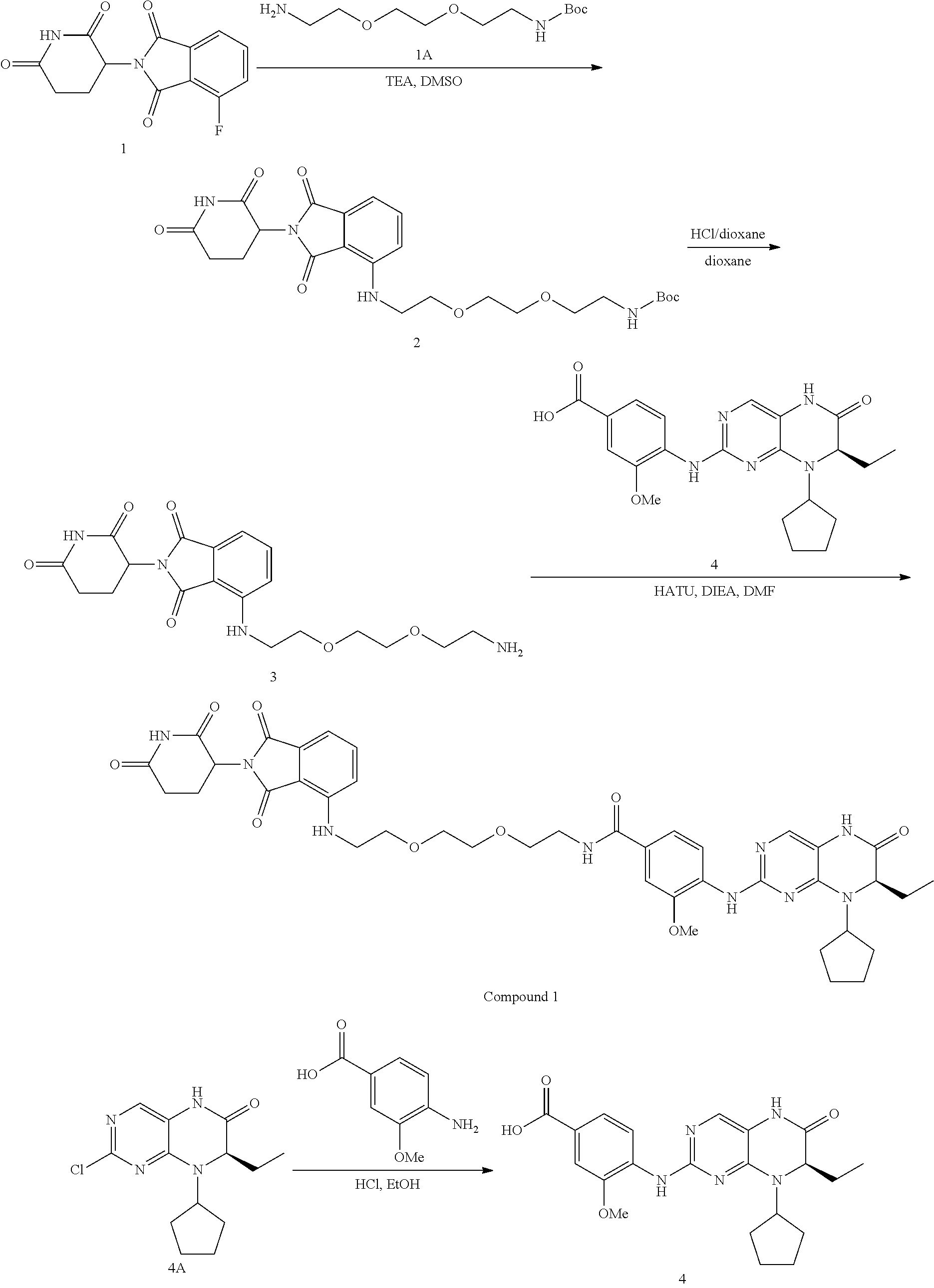

### Experimental Procedure:

### Step 1. Synthesis of (R)-4-((8-cyclopentyl-7-ethyl-6-oxo-5, 6, 7,8-tetrahydropteridin-2-yl)amino)-3-methoxybenzoic acid (4)

To a solution of (7R)-2-chloro-8-cyclopentyl-7-ethyl-5,7-dihydropteridin-6-one (500 mg, 1.78 mmol) and 4-amino-3-methoxy-benzoic acid (446.55 mg, 2.67 mmol) in EtOH (3 mL) and H₂O (9 mL) was added HCl (12 M, 371.03 μL), and then the mixture was stirred at 100° C. under N₂ atmosphere for 12 hrs. LCMS showed trace of reactant remained and 35% of desired mass was detected. The mixture was distilled under reduced pressure, then was purified by flash silica gel chromatography (ISCO®; 40 g SepaFlash® Silica Flash Column, Eluent of 5˜20% Ethyl acetate/MeOH @ 100 mL/min). The product was diluted with H₂O (20 mL), and Na₂CO₃ solution was added to adjust pH>8, then the mixture was extracted with EtOAc (20 mL×2). To the aqueous phase was added HCl solution (1 M) to adjust pH<5, then was extracted with EtOAc (20 mL×2). The combined organic layer was washed with brine (40 mL), and dried over Na₂SO₄, and then filtered and concentrated under reduced pressure to afford titled compound (57 mg, 138.53 μmol, 10.36% yield) as a brown solid. MS (M+H)⁺=412.4

### Step 2. Synthesis of tert-butyl (2-(2-(2-((2-(2,6-dioxopiperidin-3-yl)-1,3-dioxoisoindolin-4-yl)amino)ethoxy)ethoxy)ethyl)carbamate (2)

To a solution of 2-(2,6-dioxo-3-piperidyl)-4-fluoro-isoindoline-1,3-dione (3 g, 10.86 mmol) and tert-butyl N-[2-[2-(2-aminoethoxy)ethoxy]ethyl]carbamate (2.70 g, 10.86 mmol) in DMSO (50 mL) was added TEA (2.20 g, 21.72 mmol, 3.02 mL). The mixture was stirred at 90° C. for 16 hrs. TLC (PE/EtOAc=10/1) showed one main spot was formed. The reactant was cooled to room temperature, then diluted with H₂O (200 mL) and extracted with EtOAc (200 mL×3). The combined organic layer was dried over Na₂SO₄, then filtered and concentrated under reduced pressure. The residue was purified by column chromatography (SiO₂, PE/EtOAc:30%˜50%) to afford the titled compound (2.8 g, 4.22 mmol, 38.83% yield, 76% purity) as a yellow oil. MS(M+H)⁺=505.1

### Step 3. Synthesis of 4-((2-(2-(2-aminoethoxy)ethoxy)ethyl)amino)-2-(2,6-dioxopiperidin-3-yl)isoindoline-1,3-dione (3)

To a solution of tert-butyl (2-(2-(2-((2-(2,6-dioxopiperidin-3-yl)-1,3-dioxoisoindolin-4-yl)amino)ethoxy)ethoxy)ethyl)carbamate (2.8 g, 4.22 mmol, 76% purity) in dioxane (20 mL) was added HCl/dioxane (4 M, 20 mL). The mixture was stirred at 20° C. for 3 hr. LCMS showed one peak (72%) with desired mass was detected. The mixture was concentrated under reduced pressure to afford the titled compound (2.9 g, crude, HCl salt) as a yellow oil. MS(M+H)⁺=405.1

### Step 4. Synthesis of 4-(((R)-8-cyclopentyl-7-ethyl-6-oxo-5,6,7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methoxybenzamide (Compound 1)

To a solution of (R)-4-((8-cyclopentyl-7-ethyl-6-oxo-5,6,7,8-tetrahydropteridin-2-yl)amino)-3-methoxybenzoic acid (57 mg, 138.53 μmol) in DMF (3 mL) was added DIEA (107.43 mg, 831.20 μmol, 144.78 μL) and HATU (79.01 mg, 207.80 μmol), then was stirred at 20° C. for 15 minutes. Then to this mixture was added 4-((2-(2-(2-aminoethoxy)ethoxy)ethyl)amino)-2-(2,6-dioxopiperidin-3-yl)isoindoline-1,3-dione (73.29 mg, 166.24 μmol, HCl salt), then was stirred at 20° C. for 2 hrs. LCMS showed the reactant was consumed completely and 61% of desired mass was detected. The mixture was filtered, then the filtrate was purified by prep-HPLC (column: Waters Xbridge 150*25 mm*5 μm; mobile phase: [water (10 mM NH₄HCO₃)-ACN]; B %: 30%-60%, 9 min) and was lyophilized to afford the titled compound (41.1 mg, 46.88 μmol, 33.84% yield, 91% purity) as a yellow solid. MS(M+H)⁺=798.3

¹H NMR (400 MHz, DMSO-d₆) δ=11.09 (s, 1H), 10.54 (s, 1H), 8.44-8.28 (m, 2H), 7.60-7.50 (m, 3H), 7.50-7.40 (m, 2H), 7.10 (d, J=8.6 Hz, 1H), 7.02 (d, J=7.0 Hz, 1H), 6.59 (t, J=5.6 Hz, 1H), 5.06 (dd, J₁=12.8 Hz, J₂=5.4 Hz, 1H), 4.30 (q, J=8.4 Hz, 1H), 4.13 (dd, J₁=7.3 Hz, J₂=3.4 Hz, 1H), 3.92 (s, 3H), 3.65-3.60 (m, 2H), 3.60-3.50 (m, 6H), 3.47-3.39 (m, 4H), 2.93-2.83 (m, 1H), 2.63-2.52 (m, 2H), 2.07-1.97 (m, 2H), 1.91-1.88 (m, 2H), 1.85-1.73 (m, 4H), 1.71-1.65 (m, 1H), 1.65-1.54 (m, 2H), 0.81 (t, J=7.4 Hz, 3H)

### Example 2: Synthesis of 4-(((R)-8-cyclopentyl-5-methyl-6-oxo-7-propyl-5,6,7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methoxybenzamide (Compound 2)

### Synthetic Scheme:

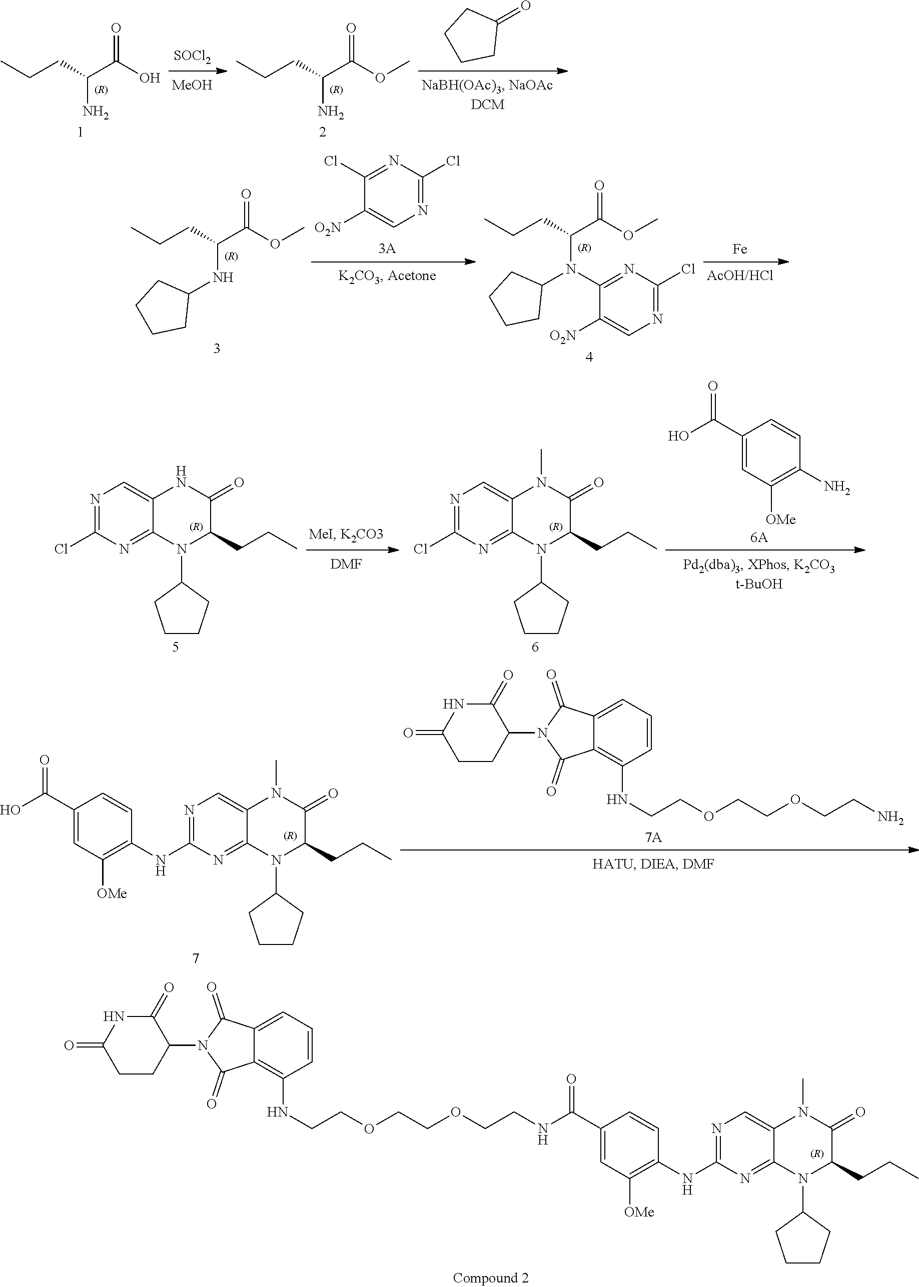

### Experimental Procedure:

### Step 1. Synthesis of (R)-methyl 2-aminopentanoate (2)

To a solution of (R)-2-aminopentanoic acid (15 g, 128.05 mmol) in MeOH (80 mL) was added SOCl₂ (30.47 g, 256.09 mmol, 18.58 mL) drop-wise, then was stirred at 70° C. for 12 hrs. TLC (Petroleum ether/Ethyl acetate=5/1) showed the starting material was consumed completely and one new spot with lower polarity was detected. The mixture was concentrated under reduced pressure to afford the titled compound (24.3 g, crude) as a colorless oil, which was used in the next step without further purification.

Step 2. Synthesis of (R)-methyl 2-(cyclopentylamino)pentanoate (3)

To a solution of (R)-methyl 2-aminopentanoate (24.3 g, 185.25 mmol) and cyclopentanone (15.58 g, 185.25 mmol, 16.40 mL) in THF (300 mL) was added NaOAc (15.20 g, 185.25 mmol), and NaBH(OAc)₃ (54.97 g, 259.35 mmol) was added slowly, then the mixture was stirred at 20° C. for 4 hrs. LCMS showed the starting material was consumed completely and desired mass was detected. The mixture was diluted with H₂O (500 mL), then extracted with EtOAc (300 mL×4). The combined organic layer was washed with brine (500 mL), then dried over Na₂SO₄, and filtered. The filtrate was concentrated under reduced pressure to afford 22.03 g crude product. 21.5 g crude product was diluted with H₂O (50 mL), then adjusted to pH>7 with saturated NaHCO₃ solution. The mixture was extracted with EtOAc (150 mL×3). The combined organic layer was washed with brine (150 mL), then dried over Na₂SO₄, and filtered and concentrated under reduced pressure to afford the titled compound (17.05 g) as a brown oil, which was used in the next step without further purification. MS(M+H)⁺=200.2

### Step 3. Synthesis of (R)-methyl 2-((2-chloro-5-nitropyrimidin-4-yl) (cyclopentyl)amino)pentanoate (4)

To a solution of methyl (R)-methyl 2-(cyclopentylamino)pentanoate (16.5 g, 82.79 mmol) in ACETONE (250 mL) was added K₂CO₃ (40.05 g, 289.78 mmol), then a solution of 2,4-dichloro-5-nitro-pyrimidine (27.30 g, 140.75 mmol) in ACETONE (50 mL) was added. The mixture was stirred at 20° C. for 16 hrs. LCMS showed trace of the starting material remained and 45% of desired mass was detected. The mixture was diluted with H₂O (300 mL) and extracted with EtOAc (400 mL×4). The combined organic layer was dried over Na₂SO₄, then filtered and concentrated under reduced pressure. The residue was purified by column chromatography (SiO₂, Petroleum ether/Ethyl acetate=100/1 to 70/1) to afford the light yellow titled compound (10.3 g, 28.87 mmol, 34.87% yield). MS(M+H)⁺=356.9

### Step 4. Synthesis of (R)-2-chloro-8-cyclopentyl-7-propyl-7,8-dihydropteridin-6 (5H)-one (5)

To a solution of (R)-methyl 2-((2-chloro-5-nitropyrimidin-4-yl) (cyclopentyl)amino)pentanoate (10.3 g, 28.87 mmol) in AcOH (60 mL) was added Fe (6.45 g, 115.47 mmol) and HCl (12 M, 14.43 mL), then the mixture was stirred at 70° C. for 12 hrs. TLC (Petroleum ether:Ethyl acetate=3:1) indicated the starting material was consumed completely and one spot with higher polarity was detected. The mixture was quenched with ice water (500 mL), and then Na₂CO₃ solution was added to adjust pH>7, and the mixture was extracted with EtOAc (300 mL×3). The combined organic layer was washed with brine (400 mL), and dried over Na₂SO₄, then filtered and concentrated under reduced pressure. The residue was purified by flash silica gel chromatography (ISCO®; 40 g SepaFlash® Silica Flash Column, Eluent of 9˜60% Ethyl acetate/Petroleum ether gradient @ 100 mL/min) to afford the titled compound (6.5 g, 22.05 mmol, 76.39% yield)) as a brown solid. MS(M+H)⁺=295.8

### Step 5. Synthesis of (R)-2-chloro-8-cyclopentyl-5-methyl-7-propyl-7,8-dihydropteridin-6 (5H)-one (6)

To a solution of (R)-2-chloro-8-cyclopentyl-7-propyl-7,8-dihydropteridin-6(5H)-one (2 g, 6.78 mmol) in DMF (20 mL) was added K₂CO₃ (2.81 g, 20.35 mmol) followed by iodomethane (1.44 g, 10.18 mmol, 633.56 μL) drop-wise at 0° C. The mixture was stirred at 20° C. for 2 hrs. LCMS showed trace of the starting material remained and one peak (89%) with desired mass was detected. The reaction mixture was diluted with H₂O (150 mL), and then extracted with EtOAc (100 mL×2). The combined organic layer was washed with brine (150 mL×5), then dried over Na₂SO₄, filtered and concentrated under reduced pressure to afford the titled compound (2.2 g, crude) as a brown solid, which was used in the next step without further purification. MS(M+H)⁺=309.8

### Step 6. Synthesis of (R)-4-((8-cyclopentyl-5-methyl-6-oxo-7-propyl-5, 6, 7,8-tetrahydropteridin-2-yl)amino)-3-methoxybenzoic acid (7)

To a solution of (R)-2-chloro-8-cyclopentyl-5-methyl-7-propyl-7,8-dihydropteridin-6 (5H)-one (500 mg, 1.62 mmol), 4-amino-3-methoxybenzoic acid (270.66 mg, 1.62 mmol), K₂CO₃ (671.33 mg, 4.86 mmol) and XPhos (92.62 mg, 194.30 μmol) in t-BuOH (5 mL) was added Pd₂(dba)₃ (148.27 mg, 161.91 μmol), then the mixture was stirred at 100° C. for 16 hrs under N₂ atmosphere. LCMS showed the starting material was consumed completely and 55% of desired mass was detected. The mixture was concentrated under reduced pressure, then the residue was diluted with H₂O (80 mL), and then washed with EtOAc (60 mL×2). To the aqueous phase was added HCl solution (1M) to adjust pH<5, then the mixture was extracted with EtOAc (20 mL×2), and the combined organic layer was dried over Na₂SO₄, then filtered and concentrated under reduced pressure to afford the titled compound (550 mg, 1.25 mmol, 77.29% yield) as a brown solid, which was used in the next step without further purification. MS(M+H)⁺=440.0

### Step 7. Synthesis of 4-(((R)-8-cyclopentyl-5-methyl-6-oxo-7-propyl-5, 6, 7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methoxybenzamide (Compound 2)

To a solution of (R)-4-((8-cyclopentyl-5-methyl-6-oxo-7-propyl-5,6,7,8-tetrahydropteridin-2-yl)amino)-3-methoxybenzoic acid (129.60 mg, 294.87 μmol) in DMF (3 mL) were added DIEA (175.89 mg, 1.36 mmol, 237.05 μL) and HATU (129.37 mg, 340.23 μmol), then the mixture was stirred at 20° C. for 15 minutes. Then to the mixture was added 4-((2-(2-(2-aminoethoxy)ethoxy)ethyl)amino)-2-(2,6-dioxopiperidin-3-yl)isoindoline-1,3-dione (100 mg, 226.82 μmol, HCl salt), then the mixture was stirred at 20° C. for further 2 hrs. LCMS showed 45% of desired mass was detected.

The reaction mixture was combined with another batch (50 mg scale) for further purification. The combined mixture was filtered, and then the filtrate was purified by prep-HPLC (column: Phenomenex luna C₁₈ 150*25 mm*10 μm; mobile phase: [water (0.225% FA)-ACN]; B %: 19%-55%, 12 min). Then NaHCO₃ solution was added to adjust pH>7, then the mixture was extracted with DCM (20 mL×3). The organic layer was dried over Na₂SO₄, and then concentrated. The residue was diluted with H₂O (10 mL) and ACN (10 mL), and then lyophilized. The product was re-purified by prep-TLC (SiO₂, Petroleum ether/Ethyl acetate=10/1) to afford the titled compound (22.0 mg, 0.02584 mmol, 11.39% yield, 97% purity) as a yellow solid. MS(M+H)⁺=826.7

¹HNMR (400 MHz, DMSO-d₆) δ=11.09 (s, 1H), 8.51-8.27 (m, 2H), 7.85 (s, 1H), 7.60 (s, 1H), 7.55 (dd, J=8.4, 7.3 Hz, 1H), 7.52-7.43 (m, 2H), 7.10 (d, J=8.5 Hz, 1H), 7.02 (d, J=7.0 Hz, 1H), 6.59 (t, J=5.8 Hz, 1H), 5.06 (dd, J=12.8, 5.4 Hz, 1H), 4.35 (t, J=7.9 Hz, 1H), 4.28-4.25 (m, 1H), 3.93 (s, 3H), 3.65-3.60 (m, 2H), 3.59-3.53 (m, 5H), 3.46-3.39 (m, 4H), 3.24 (s, 3H), 2.94-2.82 (m, 1H), 2.61-2.55 (m, 2H), 2.06-1.98 (m, 2H), 1.94-1.84 (m, 2H), 1.81-1.77 (m, 2H), 1.70-1.56 (m, 4H), 1.30-1.13 (m, 4H), 0.82 (t, J=7.3 Hz, 3H)

### Example 3: Synthesis of 4-(((R)-8-cyclopentyl-7-ethyl-5-methyl-6-oxo-5, 6,7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methylbenzamide (Compound 3)

### Synthetic Scheme:

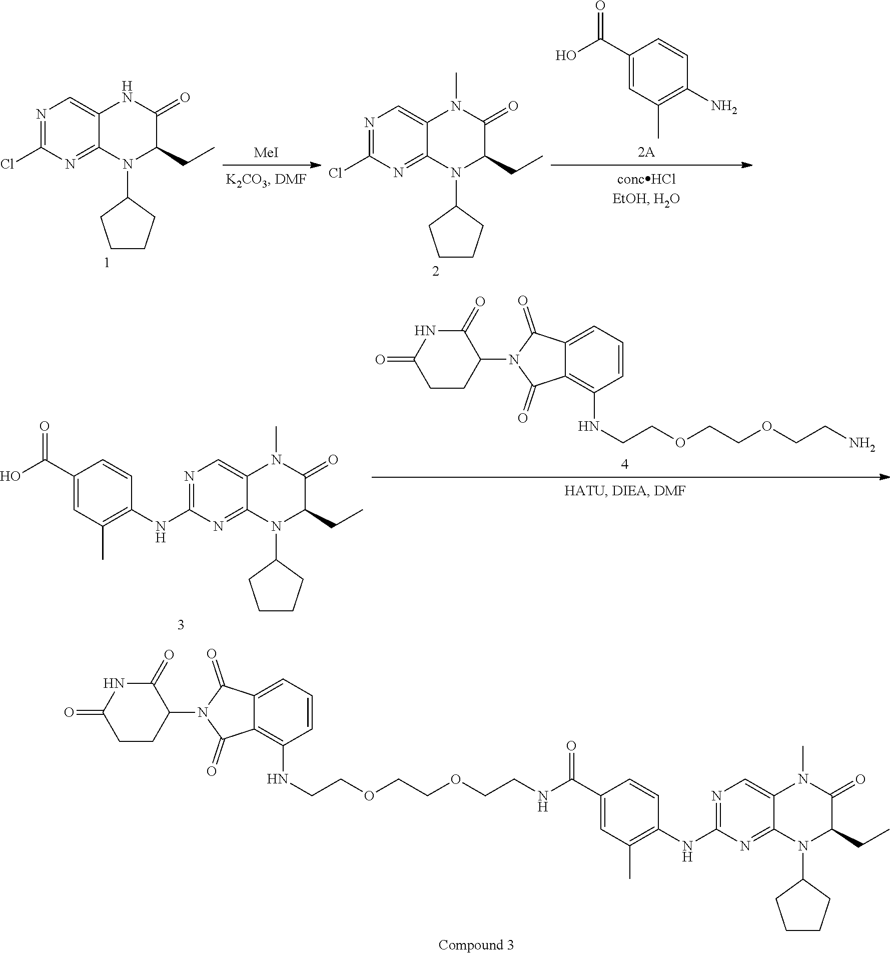

### Experimental Procedure:

### Step 1. Synthesis of (R)-2-chloro-8-cyclopentyl-7-ethyl-5-methyl-7,8-dihydropteridin-6(5H)-one (2)

To a solution of (R)-2-chloro-8-cyclopentyl-7-ethyl-7,8-dihydropteridin-6(5H)-one (3.36 g, 11.97 mmol) and K₂CO₃ (4.96 g, 35.91 mmol) in DMF (20 mL) was added iodomethane (2.55 g, 17.96 mmol, 1.12 mL) drop-wise, then the mixture was stirred at 15° C. for 12 hrs. LCMS showed the starting material was consumed completely and 82% of desired mass was detected. The mixture was quenched by addition of H₂O (200 mL) at 0° C., and extracted with EtOAc (150 mL×3). The combined organic layer was washed with brine (150 mL×5), then dried over Na₂SO₄, filtered and concentrated under reduced pressure. The residue was purified by flash silica gel chromatography (20 g SepaFlash® Silica Flash Column, Eluent of 9˜50% Ethyl acetate/Petroleum ether gradient @100 mL/min) to afford the titled compound (2.67 g, 9.06 mmol, 75.67% yield) as a white solid. MS(M+H)⁺=295.1

### Step 2. Synthesis of (R)-4-((8-cyclopentyl-7-ethyl-5-methyl-6-oxo-5, 6, 7,8-tetrahydropteridin-2-yl)amino)-3-methylbenzoic acid (3)

To a solution of (R)-2-chloro-8-cyclopentyl-7-ethyl-7,8-dihydropteridin-6(5H)-one (200 mg, 678.47 μmol) and 4-amino-3-methylbenzoic acid (123.07 mg, 814.17 μmol) in EtOH (3 mL) and H₂O (10 mL) was added HCl (12 M, 119.86 μL), then the mixture was stirred at 100° C. for 16 hours. LCMS showed 14% of the starting material remained and 33% of desired mass was detected. The mixture was concentrated under reduced pressure. The residue was triturated with a EtOAc (10 mL) and ACN (10 mL) for 5 minutes, then the suspension was filtered. The filter cake was washed with EtOAc (10 mL), and then dried to afford the off-white titled compound (100 mg, 244.21 μmol, 35.99% yield). MS(M+H)⁺=410.2

### Step 3. Synthesis of 4-(((R)-8-cyclopentyl-7-ethyl-5-methyl-6-oxo-5, 6, 7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methylbenzamide (Compound 3)

To a solution of (R)-4-((8-cyclopentyl-7-ethyl-5-methyl-6-oxo-5,6,7,8-tetrahydropteridin-2-yl)amino)-3-methylbenzoic acid (90 mg, 219.79 μmol) in DMF (1.5 mL) were added HATU (125.36 mg, 329.69 μmol) and DIEA (113.63 mg, 879.16 μmol, 153.13 μL), then the mixture was stirred at 15° C. for 15 minutes. To this mixture was added 4-((2-(2-(2-aminoethoxy)ethoxy)ethyl)amino)-2-(2,6-dioxopiperidin-3-yl)isoindoline-1,3-dione (116.28 mg, 263.75 μmol, HCl salt), then the resulting mixture was stirred at 15° C. for 1 hour. LCMS showed the starting material was consumed completely and 53% of desired mass was detected. To the mixture was added CH₃COOH to adjust pH<7. The resulting mixture was purified by prep-HPLC (column: Waters Xbridge 150*25 mm*5 μm; mobile phase: [water (10 mM NH₄HCO₃)-ACN]; B %: 38%-68%, 10 min) and prep-HPLC (column: Phenomenex Synergi C₁₈ 150*25 mm*10 μm; mobile phase: [water (0.225% FA)-ACN]; B %: 14%-53%, 13 min), then lyophilized to afford the titled compound (31.5 mg, 38.79 μmol, 17.65% yield, 98% purity) as a yellow solid. MS(M+H)⁺=796.5

¹H NMR (400 MHz, DMSO-d₆) δ=11.08 (s, 1H), 8.29 (t, J=5.5 Hz, 1H), 8.09 (s, 1H), 7.84 (d, J=8.3 Hz, 1H), 7.78 (s, 1H), 7.68 (s, 1H), 7.66-7.61 (m, 1H), 7.60-7.48 (m, 1H), 7.11 (d, J=8.6 Hz, 1H), 7.03 (d, J=7.0 Hz, 1H), 6.60 (t, J=5.6 Hz, 1H), 5.06 (dd, J=5.4, 12.8 Hz, 1H), 4.32-4.05 (m, 2H), 3.62 (d, J=5.3 Hz, 2H), 3.60-3.48 (m, 6H), 3.47-3.38 (m, 4H), 3.23 (s, 3H), 2.93-2.82 (m, 1H), 2.69-2.63 (m, 2H), 2.28 (s, 3H), 2.07-1.97 (m, 1H), 1.93-1.90 (m, 1H), 1.86-1.78 (m, 2H), 1.76-1.73 (m, 2H), 1.68-1.56 (m, 3H), 1.54-1.40 (m, 2H), 0.77 (t, J=7.5 Hz, 3H)

### Example 4: Synthesis of 4-(((R)-8-cyclopentyl-7-ethyl-5-methyl-6-oxo-5, 6,7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)benzamide (Compound 4)

### Synthetic Scheme:

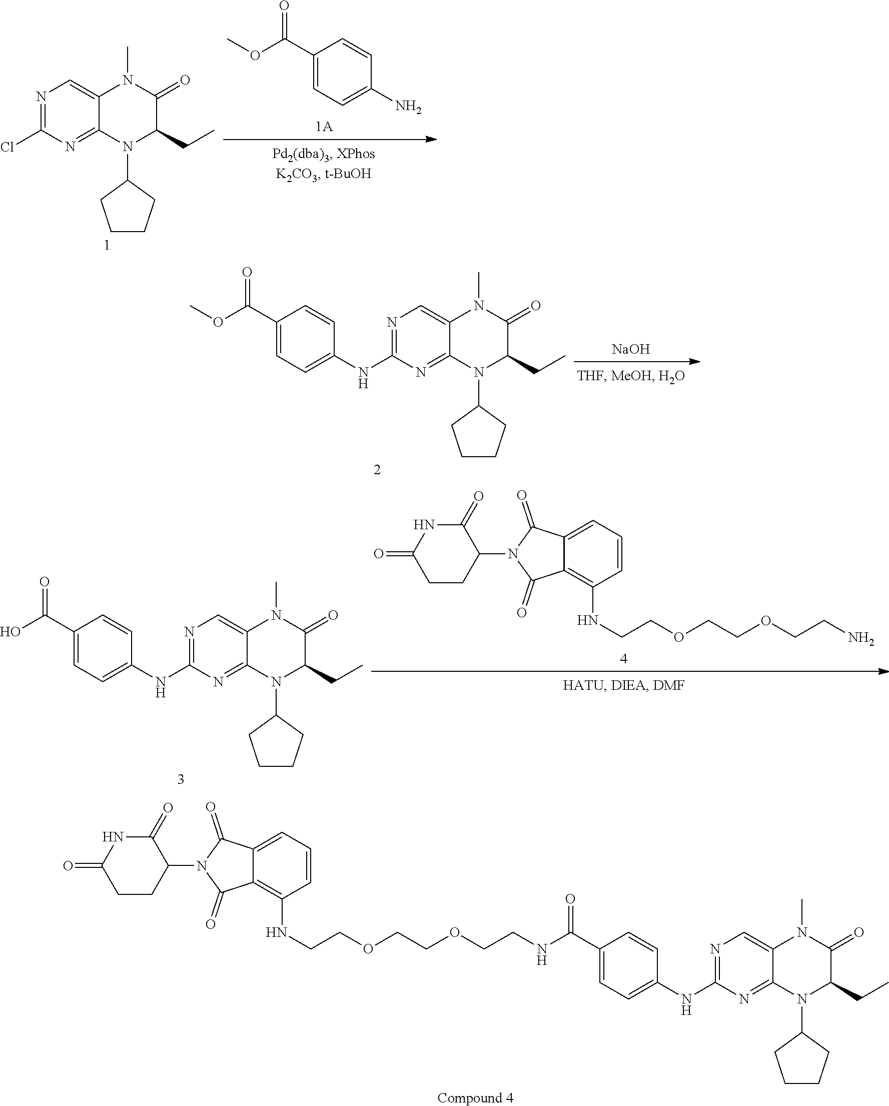

### Experimental Procedure:

### Step 1. Synthesis of (R)-2-chloro-8-cyclopentyl-7-ethyl-5-methyl-7,8-dihydropteridin-6(5H)-one (2)

To a solution of (R)-2-chloro-8-cyclopentyl-7-ethyl-5-methyl-7,8-dihydropteridin-6 (5H)-one (200 mg, 678.47 μmol), methyl 4-aminobenzoate (123.07 mg, 814.17 μmol), Pd₂(dba)₃ (62.13 mg, 67.85 μmol), XPhos (48.52 mg, 101.77 μmol) and K₂CO₃ (375.08 mg, 2.71 mmol) in t-BuOH (6 mL) was stirred at 100° C. for 6 hrs under N₂ atmosphere. LCMS showed the starting material was consumed completely and 73% of desired mass was detected. The mixture was concentrated under reduced pressure, then the residue was purified by flash silica gel chromatography (20 g SepaFlash® Silica Flash Column, Eluent of 17˜60% Ethyl acetate/Petroleum ether gradient @ 60 mL/min) to afford the titled compound (240 mg, 586.11 μmol, 86.39% yield) as a red solid. MS(M+H)⁺=410.2

### Step 2. (R)-4-((8-cyclopentyl-7-ethyl-5-methyl-6-oxo-5, 6, 7,8-tetrahydropteridin-2-yl)amino)benzoic acid (3)

To a solution of (R)-methyl 4-((8-cyclopentyl-7-ethyl-5-methyl-6-oxo-5,6,7,8-tetrahydropteridin-2-yl)amino)benzoate (230 mg, 561.69 μmol) in MeOH (2 mL) and THF (2 mL) was added a solution of NaOH (112.33 mg, 2.81 mmol) in H₂O (2 mL), and the mixture was stirred at 15° C. for 16 hrs. LCMS showed trace of the starting material remained and 93% of desired mass was detected. The mixture was concentrated under reduced pressure to remove most of the solvent, and HCl solution (12 M) was added to adjust pH<4. The mixture was filtered and the filtrate was concentrated under reduced pressure, then the residue was triturated for 5 minutes with addition of EtOAc (10 mL). The suspension was filtered, and then the filter cake was washed with EtOAc (5 mL) and dried to afford the off-white titled compound (220 mg, 556.32 μmol, 99.04% yield). MS(M+H)⁺=396.0

### Step 3. Synthesis of 4-(((R)-8-cyclopentyl-7-ethyl-5-methyl-6-oxo-5,6, 7, 8-tetrahydropteridin-2-yl)amino)-N-(2-(2-(2-((2-(2, 6-dioxopiperidin-3-yl)-1,3-dioxoisoindolin-4-yl)amino)ethoxy)ethoxy)ethyl)benzamide (Compound 4)

To a solution of (R)-4-((8-cyclopentyl-7-ethyl-5-methyl-6-oxo-5,6,7,8-tetrahydropteridin-2-yl)amino)benzoic acid (70 mg, 177.01 μmol) in DMF (2 mL) were added HATU (100.96 mg, 265.52 μmol) and DIEA (114.39 mg, 885.06 μmol, 154.16 μL), then the mixture was stirred at 15° C. for 15 minutes. To this mixture was added 4-((2-(2-(2-aminoethoxy)ethoxy)ethyl)amino)-2-(2,6-dioxopiperidin-3-yl)isoindoline-1,3-dione (109.26 mg, 247.82 μmol, HCl salt), then the resulting mixture was stirred at 15° C. for 1 hour. LCMS showed 23% of the starting material remained and 40% of desired mass was detected. To the mixture was added CH₃COOH to adjust pH<7. The resulting mixture was purified by prep-HPLC (column: Waters Xbridge 150*25 mm*5 μm; mobile phase: [water (10 mM NH₄HCO₃)-ACN]; B %: 35%-65%, 10 min) and prep-HPLC (column: Phenomenex Synergi C₁₈ 150*25 mm*10 μm; mobile phase: [water (0.225% FA)-ACN]; B %: 17%-53%, 12 min), then was lyophilized to afford the titled compound (30.4 mg, 38.10 μmol, 21.53% yield, 98% purity) as a yellow solid. MS(M+H)⁺=782.7

¹H NMR (400 MHz, DMSO-d₆) δ=11.21-10.93 (m, 1H), 9.33 (s, 1H), 8.26 (br t, J=5.6 Hz, 1H), 7.84 (s, 1H), 7.76 (q, J=9.0 Hz, 4H), 7.55 (dd, J=7.3, 8.3 Hz, 1H), 7.10 (d, J=8.6 Hz, 1H), 7.02 (d, J=7.0 Hz, 1H), 6.59 (br t, J=5.6 Hz, 1H), 5.04 (dd, J=5.0, 12.6 Hz, 1H), 4.47-4.37 (m, 1H), 4.22 (dd, J=3.5, 7.8 Hz, 1H), 3.60 (br d, J=5.4 Hz, 2H), 3.57 (br d, J=1.8 Hz, 4H), 3.52 (br t, J=6.1 Hz, 2H), 3.45-3.38 (m, 4H), 3.25 (s, 3H), 2.91-2.81 (m, 1H), 2.59 (br d, J=2.6 Hz, 2H), 2.08-1.98 (m, 2H), 1.96-1.89 (m, 1H), 1.85-1.71 (m, 5H), 1.66-1.55 (m, 3H), 0.77 (t, J=7.5 Hz, 3H)

### Example 5: Synthesis of 4-(((R)-8-cyclopentyl-7-ethyl-5-methyl-6-oxo-5, 6,7, 8-tetrahydropteridin-2-yl)amino)-N-(14-((2-(2,6-dioxopiperidin-3-yl)-1,3-dioxoisoindolin-4-yl)amino)-3,6,9,12-tetraoxatetradecyl)-3-methoxybenzamide (Compound 5)

### Synthetic Scheme:

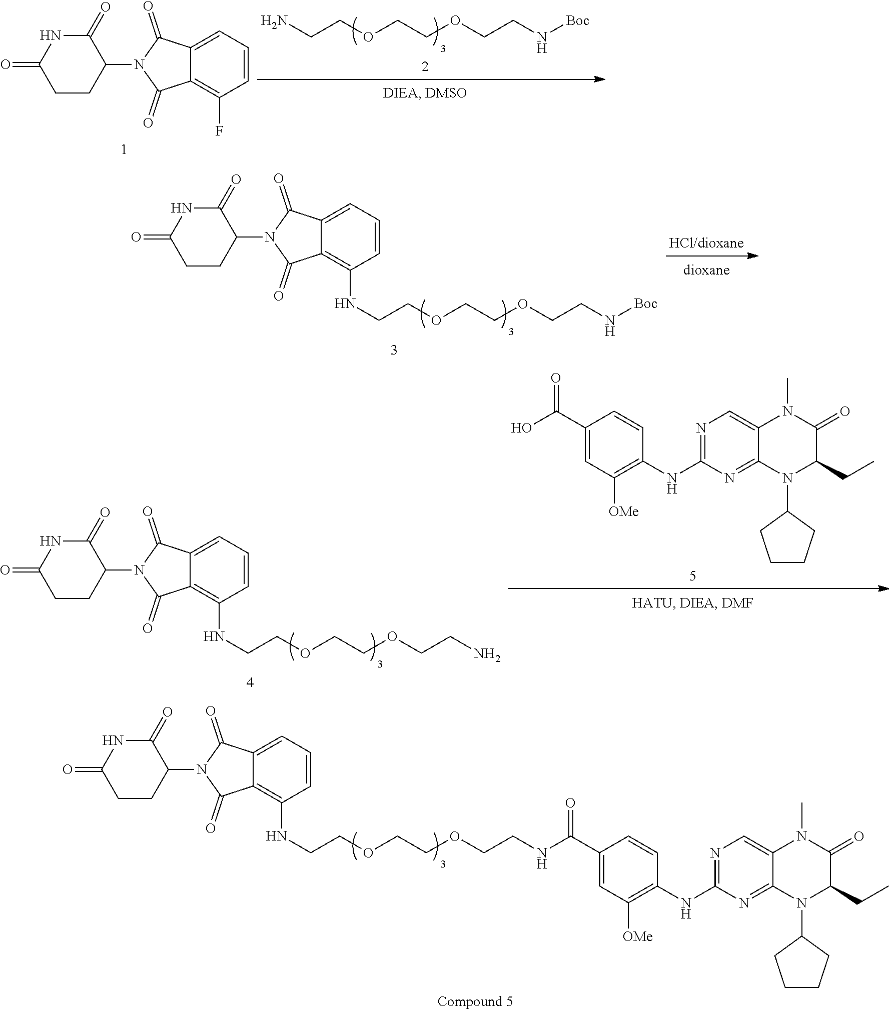

### Experimental Procedure:

### Step 1. Synthesis of tert-butyl (14-((2-(2, 6-dioxopiperidin-3-yl)-1,3-dioxoisoindolin-4-yl)amino)-3,6,9,12-tetraoxatetradecyl)carbamate (3)

A solution of 2-(2,6-dioxopiperidin-3-yl)-4-fluoroisoindoline-1,3-dione (800 mg, 2.90 mmol), tert-butyl (14-amino-3,6,9,12-tetraoxatetradecyl)carbamate (1.07 g, 3.19 mmol) and DIPEA (748.64 mg, 5.79 mmol, 1.01 mL) in DMSO (8 mL) was stirred at 80° C. for 24 hrs. LCMS showed 30% of the starting material remained and 51% of desired mass was detected. The mixture was diluted with H₂O (80 mL), then extracted with EtOAc (80 mL×3). The combined organic layer was washed with brine (150 mL×5), and dried over Na₂SO₄, and filtered, and then concentrated under reduced pressure.

The residue was purified by flash silica gel chromatography (12 g SepaFlash® Silica Flash Column, Eluent of 20˜75% Ethyl acetate/Petroleum ether gradient @ 100 mL/min) to afford the titled compound (860 mg, 1.45 mmol, 50.10% yield) as a yellow oil. MS(M+H)⁺=593.5

### Step 2. Synthesis of 4-((14-amino-3,6,9,12-tetraoxatetradecyl)amino)-2-(2, 6-dioxopiperidin-3-yl)isoindoline-1,3-dione (4)

To a solution of tert-butyl (14-((2-(2,6-dioxopiperidin-3-yl)-1,3-dioxoisoindolin-4-yl)amino)-3,6,9,12-tetraoxatetradecyl)carbamate (860 mg, 1.45 mmol) in dioxane (5 mL) was added HCl/dioxane (4 M, 15 mL), then the mixture was stirred at 15° C. for 12 hrs. LCMS showed the starting material was consumed completely and 99% of desired mass was detected. The mixture was concentrated under reduced pressure to afford the titled compound (760 mg, HCl salt) as a green oil, which was used for the next step directly. MS(M+H)⁺=493.5

### Step 3. Synthesis of 4-(((R)-8-cyclopentyl-7-ethyl-5-methyl-6-oxo-5,6, 7, 8-tetrahydropteridin-2-yl)amino)-N-(14-((2-(2, 6-dioxopiperidin-3-yl)-1,3-dioxoisoindolin-4-yl)amino)-3,6,9,12-tetraoxatetradecyl)-3-methoxybenzamide (Compound 5)

To a solution of (R)-4-((8-cyclopentyl-7-ethyl-5-methyl-6-oxo-5,6,7,8-tetrahydropteridin-2-yl)amino)-3-methoxybenzoic acid (80 mg, 188.02 μmol) in DMF (2 mL) were added HATU (107.24 mg, 282.03 μmol) and DIEA (145.80 mg, 1.13 mmol, 196.50 μL), then the mixture was stirred at 15° C. for 15 minutes. To the mixture was added 4-((14-amino-3,6,9,12-tetraoxatetradecyl)amino)-2-(2,6-dioxopiperidin-3-yl)isoindoline-1,3-dione (99.46 mg, 188.02 μmol, HCl salt), then stirred at 15° C. for 1 hour. LCMS showed the starting material was consumed completely and 69% of desired mass was detected. To the mixture was added CH₃COOH to adjust pH<7. The resulting mixture was purified by prep-HPLC (column: Waters Xbridge 150*25 mm*5 μm; mobile phase: [water (10 mM NH₄HCO₃)-ACN]; B %: 40%-70%, 9 min) and prep-HPLC (column: Phenomenex Synergi C₁₈ 150*25 mm*10 μm; mobile phase: [water (0.225% FA)-ACN]; B %: 19%-55%, 12 min), then lyophilized to afford the titled compound (45.0 mg, 48.00 μmol, 25.53% yield, 96% purity) as a yellow solid. MS(M+H)⁺=900.8

¹H NMR (400 MHz, DMSO-d₆) δ=11.09 (s, 1H), 8.49-8.32 (m, 2H), 7.84 (s, 1H), 7.62-7.53 (m, 2H), 7.53-7.44 (m, 2H), 7.12 (d, J=8.6 Hz, 1H), 7.03 (d, J=7.0 Hz, 1H), 6.58 (t, J=5.7 Hz, 1H), 5.05 (dd, J=5.3, 12.8 Hz, 1H), 4.38-4.30 (m, 1H), 4.24 (dd, J=3.5, 7.5 Hz, 1H), 3.93 (s, 3H), 3.61-3.57 (m, 2H), 3.56-3.49 (m, 10H), 3.48-3.46 (m, 4H), 3.46-3.39 (m, 5H), 3.24 (s, 3H), 2.92-2.83 (m, 1H), 2.62-2.58 (m, 1H), 2.06-1.97 (m, 2H), 1.94-1.84 (m, 2H), 1.83-1.71 (m, 4H), 1.68-1.55 (m, 3H), 0.76 (t, J=7.4 Hz, 3H)

### Example 6: Synthesis of 4-(((R)-8-cyclopentyl-7-ethyl-5-methyl-6-oxo-5, 6,7, 8-tetrahydropteridin-2-yl)amino)-N-((3S)-1-(4-(4-(2-(2, 6-dioxopiperidin-3-yl)-1,3-dioxoisoindolin-4-yl)piperazin-1-yl)-4-oxobutanoyl) pyrrolidin-3-yl)-3-methoxybenzamide (Compound 6)

### Synthetic Scheme:

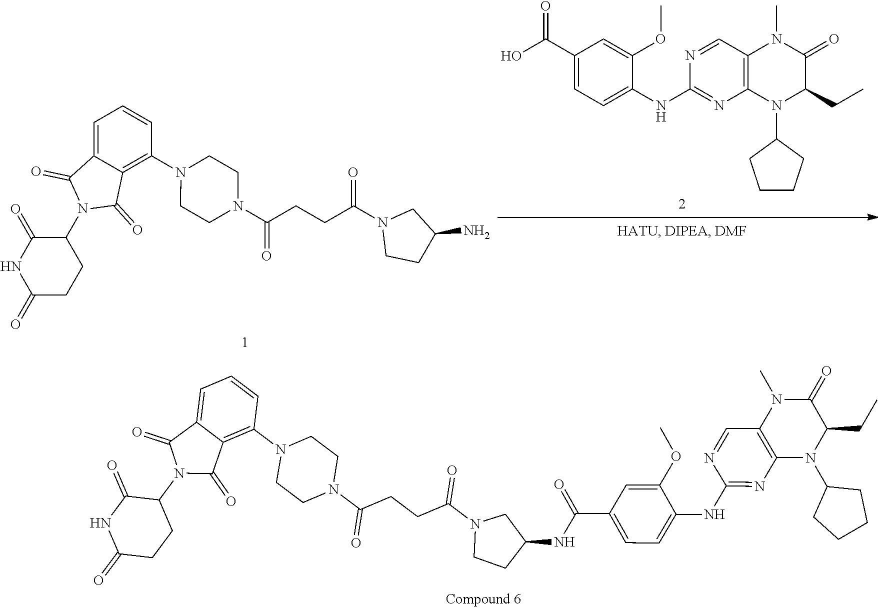 [SMILES: N[C@H]1CCN(C(=O)CCC(=O)N2CCN(c3cccc4c3C(=O)N(C3CCC(=O)NC3=O)C4=O)CC2)C1 ; CC[C@@H]1C(=O)N(C)c2cnc(Nc3ccc(C(=O)O)cc3OC)nc2N1C1CCCC1 ; CC[C@@H]1C(=O)N(C)c2cnc(Nc3ccc(C(=O)N[C@H]4CCN(C(=O)CCC(=O)N5CCN(c6cccc7c6C(=O)N(C6CCC(=O)NC6=O)C7=O)CC5)C4)cc3OC)nc2N1C1CCCC1]

### Experimental Procedure:

### Step 1. Synthesis of 4-(((R)-8-cyclopentyl-7-ethyl-5-methyl-6-oxo-5,6, 7, 8-tetrahydropteridin-2-yl)amino)-N-((3S)-1-(4-(4-(2-(2, 6-dioxopiperidin-3-yl)-1, 3-dioxoisoindolin-4-yl)piperazin-1-yl)-4-oxobutanoyl)pyrrolidin-3-yl)-3-methoxybenzamide (Compound 6)

To a solution of (R)-4-((8-cyclopentyl-7-ethyl-5-methyl-6-oxo-5,6,7,8-tetrahydropteridin-2-yl)amino)-3-methoxybenzoic acid (77.78 mg, 182.81 μmol) in DMF (2 mL) were added HATU (139.02 mg, 365.63 μmol) and DIPEA (94.51 mg, 731.26 μmol, 127.37 μL), then the mixture was stirred at 25° C. for 10 min. To the mixture was added 4-(4-(4-((S)-3-aminopyrrolidin-1-yl)-4-oxobutanoyl)piperazin-1-yl)-2-(2,6-dioxopiperidin-3-yl)isoindoline-1,3-dione (100 mg, 182.81 μmol, HCl salt), then stirred at 25° C. for 1 hour. LCMS showed peak (61%) with desired mass was detected. The mixture was purified by prep-HPLC (column: Phenomenex Synergi C₁₈ 150*25 mm*10 um; mobile phase: [water (0.225% FA)-ACN]; B %: 16%-46%, 10 min) and prep-HPLC (column: Waters Xbridge 150*25 mm*5 μm; mobile phase: [water (10 mM NH₄HCO₃)-ACN]; B %: 34%-64%, 10 min), and then lyophilized to afford the titled compound (39.2 mg, 42.40 μmol, 55.61% yield, 99.3% purity) as a yellow solid. MS(M+H)⁺=918.1

¹H NMR (400 MHz, DMSO-d₆) δ=11.23-10.84 (s, 1H), 8.46-8.32 (m, 2H), 7.85 (s, 1H), 7.75-7.69 (m, 1H), 7.61 (d, J=2.4 Hz, 1H), 7.53-7.47 (m, 2H), 7.42-7.33 (m, 2H), 5.13-5.05 (m, 1H), 4.56-4.31 (m, 2H), 4.27-4.20 (m, 1H), 3.95 (s, 3H), 3.89-3.75 (m, 1H), 3.72-3.39 (m, 7H), 3.37-3.28 (m, 1H), 3.28-3.17 (m, 5H), 2.93-2.81 (m, 1H), 2.65-2.52 (m, 7H), 2.28-2.07 (m, 1H), 2.06-1.86 (m, 5H), 1.83-1.72 (m, 4H), 1.68-1.57 (m, 3H), 0.76 (t, J=7.4 Hz, 3H)

### Example 7: Synthesis of 4-(((R)-8-cyclopentyl-7-ethyl-5-methyl-6-oxo-5, 6,7, 8-tetrahydropteridin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 7)

### Synthetic Scheme:

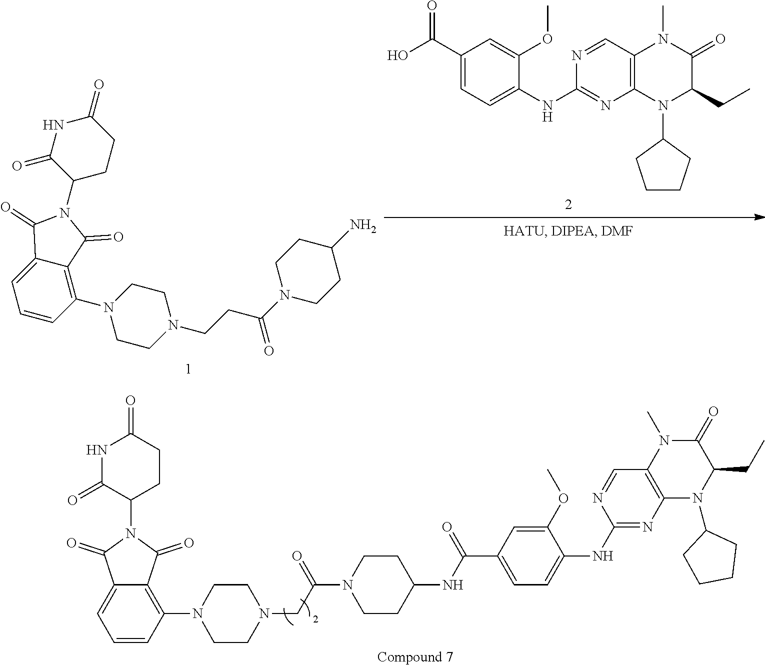

### Experimental Procedure:

### Step 1. Synthesis of 4-(((R)-8-cyclopentyl-7-ethyl-5-methyl-6-oxo-5,6, 7, 8-tetrahydropteridin-2-yl)amino)-N-(1-(3-(4-(2-(2, 6-dioxopiperidin-3-yl)-1,3-dioxoisoindolin-4-yl)piperazin-1-yl)propanoyl)piperidin-4-yl)-3-methoxybenzamide (Compound 7)

To a solution of (R)-4-((8-cyclopentyl-7-ethyl-5-methyl-6-oxo-5,6,7,8-tetrahydropteridin-2-yl)amino)-3-methoxybenzoic acid (68 mg, 159.82 μmol) in DMF (2 mL) were added HATU (91.15 mg, 239.73 μmol) and DIPEA (61.97 mg, 479.46 μmol, 83.51 μL), then the mixture was stirred at 30° C. for 10 min. To the mixture was added 4-(4-(3-(4-aminopiperidin-1-yl)-3-oxopropyl)piperazin-1-yl)-2-(2,6-dioxopiperidin-3-yl)isoindoline-1,3-dione (95.23 mg, 191.78 μmol), then stirred at 30° C. for 12 hrs. LCMS showed the starting material was consumed completely, and a main peak with desired mass was detected. The solution was concentrated under reduced pressure, then purified by prep-HPLC (column: Waters Xbridge 150*25 mm*5 μm; mobile phase: [water (10 mM NH₄HCO₃)-ACN]; B %: 36%-66%, 10 min) and lyophilized to afford the titled compound (29.2 mg, 29.39 μmol, 18.39% yield, 91% purity) as yellow solid. MS (M+H)⁺=904.4 ¹H NMR (400 MHz, DMSO-d₆) δ=11.18-10.93 (m, 1H), 8.42 (d, J=9.0 Hz, 1H), 8.11 (d, J=7.9 Hz, 1H), 7.85 (s, 1H), 7.70 (t, J=7.8 Hz, 1H), 7.60 (s, 1H), 7.52-7.45 (m, 2H), 7.35 (t, J=7.2 Hz, 2H), 5.09 (dd, J=5.3, 12.9 Hz, 1H), 4.48-4.30 (m, 2H), 4.24 (dd, J=3.5, 7.6 Hz, 1H), 4.13-3.96 (m, 2H), 3.94 (s, 3H), 3.31-3.30 (m, 4H), 3.25 (s, 3H), 3.19-3.04 (m, 1H), 2.93-2.80 (m, 1H), 2.70-2.54 (m, 12H), 2.11-1.97 (m, 2H), 1.95-1.72 (m, 8H), 1.65-1.58 (m, 2H), 1.54-1.31 (m, 2H), 0.76 (t, J=7.5 Hz, 3H)

### Example 8: Synthesis of 4-(((R)-8-cyclopentyl-7-ethyl-5-methyl-6-oxo-5, 6,7, 8-tetrahydropteridin-2-yl)amino)-N-(1-(4-((2-(2,6-dioxopiperidin-3-yl)-1,3-dioxoisoindolin-4-yl)amino) butanoyl) piperidin-4-yl)-3-methoxybenzamide (Compound 8)

### Synthetic Scheme:

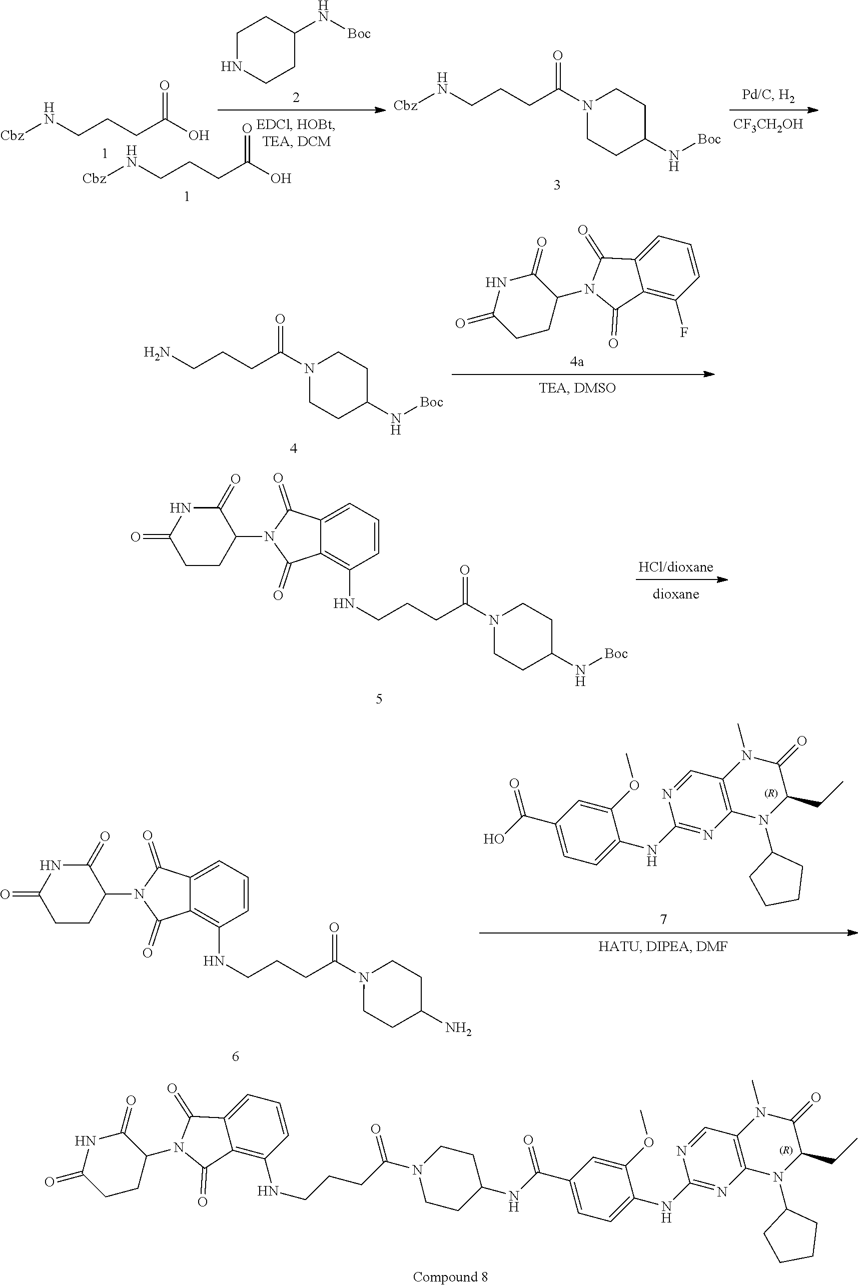

### Experimental Procedure:

### Step 1. Synthesis of tert-butyl N-[1-[4-(benzyloxycarbonylamino)butanoyl]-4-piperidyl]carbamate (3)

To a solution of 4-(((benzyloxy)carbonyl)amino)butanoic acid (1.5 g, 6.32 mmol) and tert-butyl piperidin-4-ylcarbamate (1.39 g, 6.95 mmol) in DCM (20 mL) were added HOBt (1.03 g, 7.59 mmol), EDCI (1.45 g, 7.59 mmol) and TEA (1.92 g, 18.97 mmol, 2.64 mL), then the mixture was stirred at 25° C. for 16 hrs. LCMS showed peak (80%) with desired mass was detected. The mixture was concentrated under reduced pressure, then Ethyl acetate (50 mL) was added, and stirred for 1 hour. The mixture was filtered, then the filter cake was collected and dried under reduced pressure to afford the titled compound (3.1 g, crude) as a white solid, which was used for the next step directly. MS(M+H)⁺=420.2

### Step 2. Synthesis of tert-butyl (1-(4-aminobutanoyl)piperidin-4-yl)carbamate (4)

To a solution of N-[1-[4-(benzyloxycarbonylamino)butanoyl]-4-piperidyl]carbamate (0.2 g, 476.74 μmol) in 2,2,2-trifluoroethanol (10 mL) was added Pd/C (100 mg, 476.74 μmol, 10% purity) under N₂ atmosphere. The mixture was stirred under H₂ atmosphere at 30° C. for 12 hrs. LCMS showed the starting material was consumed completely and one peak (53%) with desired mass was detected. The mixture was filtered through a celite pad, then the filtrate was concentrated to afford the titled compound (130 mg, crude) as a white solid, which was used for the next step directly. MS(M+H)⁺=286.2

### Step 3. Synthesis of tert-butyl (1-(4-((2-(2, 6-dioxopiperidin-3-yl)-1,3-dioxoisoindolin-4-yl)amino) butanoyl)piperidin-4-yl)carbamate (5)

A solution of 2-(2,6-dioxopiperidin-3-yl)-4-fluoroisoindoline-1,3-dione (125.83 mg, 455.53 μmol), tert-butyl (1-(4-aminobutanoyl)piperidin-4-yl)carbamate (130 mg, 455.53 μmol) and TEA (138.29 mg, 1.37 mmol, 190.21 μL) in DMSO (3 mL) was stirred at 100° C. for 12 hrs. LCMS showed the starting material was consumed completely, and one peak (33%) with desired mass was detected. The mixture was poured into water (20 mL) and extracted with ethyl acetate (20 mL×3). The combined organic layer was washed with brine (10 mL×3), then dried over Na₂SO₄, and filtered and concentrated under reduced pressure. The residue was purified by flash silica gel chromatography (4 g SepaFlash® Silica Flash Column, Eluent of 0˜100% Ethyl acetate/Petroleum ether gradient @ 65 mL/min) to afford the titled compound (83 mg, 151.72 μmol, 33.31% yield, 99% purity) as yellow oil, which was used for the next step directly. MS(M+H)⁺=542.1

### Step 4. Synthesis of 4-((4-(4-aminopiperidin-1-yl)-4-oxobutyl)amino)-2-(2, 6-dioxopiperidin-3-yl)isoindoline-1, 3-dione (6)

To a solution of tert-butyl (1-(4-((2-(2,6-dioxopiperidin-3-yl)-1,3-dioxoisoindolin-4-yl)amino)butanoyl)piperidin-4-yl)carbamate (83 mg, 153.25 μmol) in dioxane (1 mL) was added HCl/dioxane (4 M, 5 mL) at 25° C., then the mixture was stirred at 25° C. for 0.5 hr. LCMS showed the starting material was consumed completely and one peak (92%) with desired mass was detected. The mixture was concentrated under reduced pressure to afford the titled compound (73 mg, 145.10 μmol, 94.68% yield, 95% purity, HCl salt) as a yellow solid, which was used for the next step directly. MS(M+H)⁺=442.3

### Step 5. Synthesis of 4-(((R)-8-cyclopentyl-7-ethyl-5-methyl-6-oxo-5,6, 7, 8-tetrahydropteridin-2-yl)amino)-N-(1-(4-((2-(2, 6-dioxopiperidin-3-yl)-1,3-dioxoisoindolin-4-yl)amino)butanoyl)piperidin-4-yl)-3-methoxybenzamide (Compound 8)

To a solution of (R)-4-((8-cyclopentyl-7-ethyl-5-methyl-6-oxo-5,6,7,8-tetrahydropteridin-2-yl)amino)-3-methoxybenzoic acid (68 mg, 159.82 μmol) in DMF (2 mL) were added HATU (91.15 mg, 239.73 μmol) and DIPEA (61.97 mg, 479.46 μmol, 83.51 μL), then the mixture was stirred at 25° C. for 10 minutes. To the mixture was added 4-[[4-(4-amino-1-piperidyl)-4-oxo-butyl]amino]-2-(2,6-dioxo-3-piperidyl) isoindoline-1,3-dione (70 mg, 146.46 μmol, HCl salt), then stirred at 25° C. for 12 hrs. LCMS showed the starting material was consumed completely and a main peak with desired mass was detected. The mixture was concentrated under reduced pressure, then purified by prep-HPLC (column: Waters Xbridge 150*25 mm*5 μm; mobile phase: [water (10 mM NH₄HCO₃)-ACN]; B %: 40%-70%, 10 min), then lyophilized to afford the titled compound (34.1 mg, 38.96 μmol, 24.38% yield, 98% purity) as a yellow solid. MS (M+H)⁺=849.3

¹H NMR (400 MHz, DMSO-d₆) δ=11.21-10.95 (m, 1H), 8.43 (d, J=9.0 Hz, 1H), 8.10 (d, J=7.8 Hz, 1H), 7.85 (s, 1H), 7.65-7.56 (m, 2H), 7.53-7.43 (m, 2H), 7.20 (d, J=8.6 Hz, 1H), 7.03 (d, J=7.0 Hz, 1H), 6.69 (t, J=6.1 Hz, 1H), 5.06 (dd, J=5.4, 12.9 Hz, 1H), 4.47-4.31 (m, 2H), 4.24 (dd, J=3.5, 7.5 Hz, 1H), 4.12-4.00 (m, 1H), 3.95 (s, 3H), 3.92-3.85 (m, 1H), 3.41-3.33 (m, 1H), 3.33-3.28 (m, 1H), 3.26 (s, 3H), 3.13 (br t, J=12.3 Hz, 1H), 2.99-2.82 (m, 1H), 2.78-2.64 (m, 1H), 2.64-2.53 (m, 2H), 2.44 (br t, J=7.0 Hz, 2H), 2.09-1.97 (m, 2H), 1.95-1.71 (m, 10H), 1.70-1.55 (m, 3H), 1.53-1.33 (m, 2H), 0.77 (t, J=7.4 Hz, 3H)

### Example 9: Synthesis of 4-(((R)-8-cyclohexyl-5,7-dimethyl-6-oxo-5,6,7,8-tetrahydropteridin-2-yl)amino)-N-(2-(2-(2-((2-(2,6-dioxopiperidin-3-yl)-1,3-dioxoisoindolin-4-yl)amino)ethoxy)ethoxy)ethyl)-3-methoxybenzamide (Compound 9)

### Synthetic Scheme:

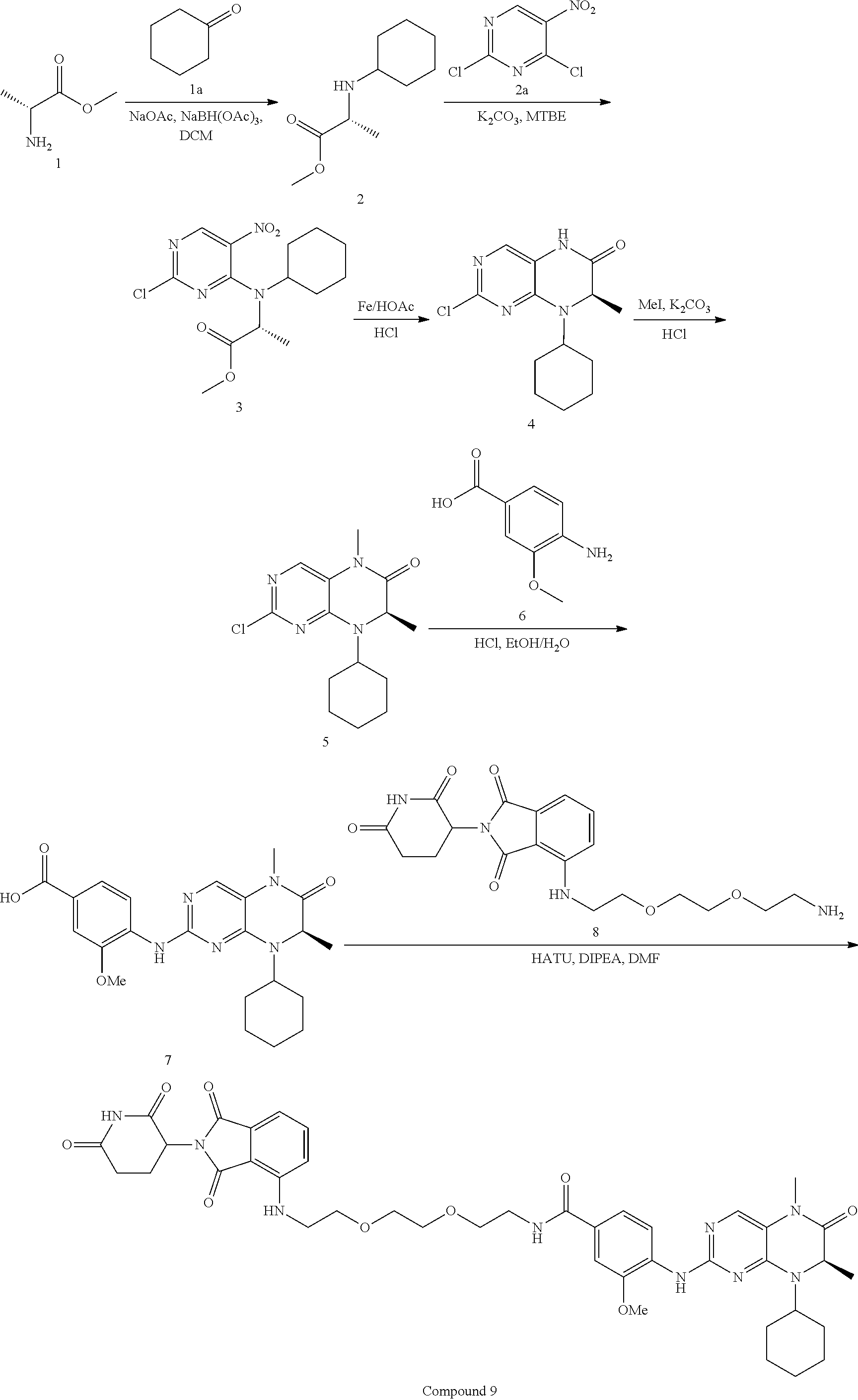

### Experimental Procedure:

### Step 1. Synthesis of (R)-methyl 2-(cyclohexylamino)propanoate (2)

To a solution of (R)-methyl 2-aminopropanoate (25 g, 179.11 mmol, HCl salt) and cyclohexanone (19.34 g, 197.02 mmol, 20.42 mL) in DCM (200 mL) were added NaOAc (14.69 g, 179.11 mmol) and NaBH(OAc)₃ (56.94 g, 268.66 mmol) at 0° C., then the mixture was stirred at 20° C. for 12 hrs. LCMS showed the desired mass was formed. The mixture was diluted with saturated Na₂CO₃ (100 mL), then extracted with DCM (100 mL×3). The combined organic layer was dried over Na₂SO₄, then filtered. The filtrate was concentrated under reduced pressure to afford the titled compound (31 g, crude) as a yellow solid, which was used for the next step directly. MS(M+H)⁺=186.3

### Step 2. Synthesis of (R)-methyl 2-((2-chloro-5-nitropyrimidin-4-yl) (cyclohexyl)amino)propanoate (3)

To a solution of (R)-methyl 2-(cyclohexylamino)propanoate (23.4 g, 126.31 mmol) in MTBE (400 mL) was added 2,4-dichloro-5-nitropyrimidine (39.20 g, 202.09 mmol) slowly at −10° C. Then a solution of K₂CO₃ (26.19 g, 189.46 mmol) in H₂O (200 mL) was added drop-wise while maintaining the temperature below 0° C. Then the mixture was warmed to 15° C. and stirred for 16 hrs. LCMS showed 21% of the starting material and peak (34%) of desired mass was detected. Another portion of 2,4-dichloro-5-nitropyrimidine (19.60 g, 101.05 mmol) was added at −10° C. Then a solution of K₂CO₃ (13.97 g, 101.05 mmol) in H₂O (50 mL) was added drop-wise while maintaining the temperature below 0° C., and then stirred at 35° C. for 24 hrs. LCMS showed 16% of starting material and peak (56%) with desired mass was detected. The mixture was filtered, then the filter cake was collected and dried in vacuo. The filtrate was extracted with ethyl acetate (100 mL×3), then the combine organic layer was dried over Na₂SO₄ and concentrated. The collected residue was treated with Petroleum ether:Ethyl acetate (10:1, 100 mL) and stirred for 1 hr, then filtered. The filter cake was collected and dried to afford the titled compound (23 g, 67.10 mmol, 53.12% yield) as a yellow solid, which was used for the next step directly. MS(M+H)⁺=343.0

### Step 3. Synthesis of (R)-2-chloro-8-cyclohexyl-7-methyl-7, 8-dihydropteridin-6 (5H)-one (4)

To a solution of (R)-methyl 2-((2-chloro-5-nitropyrimidin-4-yl) (cyclohexyl)amino)propanoate (22 g, 64.18 mmol) in AcOH (250 mL) were added Fe (21.51 g, 385.09 mmol) and con.HCl (39.00 g, 385.09 mmol, 38.24 mL, 36% purity), then the mixture was stirred at 85° C. for 12 hrs. LCMS showed the starting material was consumed completely and a main peak with desired mass was detected. The mixture was filtered, then the filtrate was diluted with H₂O (50 mL), and then refiltered, and filter cake was collected and dried. The combined residue was treated with Petroleum ether:Ethyl acetate=1:1 (100 mL) and stirred for 1 h, then filtered. The filter cake was collected and dried to afford the titled compound (16.3 g, 57.48 mmol, 89.55% yield, 99% purity) as a gray solid, which was used for the next step directly. MS(M+H)⁺=281.1

### Step 4. Synthesis of (R)-2-chloro-8-cyclohexyl-5, 7-dimethyl-7, 8-dihydropteridin-6 (5H)-one (5)

To a solution of (R)-2-chloro-8-cyclohexyl-7-methyl-7,8-dihydropteridin-6(5H)-one (0.5 g, 1.78 mmol) in DMF (5 mL) were added MeI (278.06 mg, 1.96 mmol, 121.96 μL) and K₂CO₃ (492.27 mg, 3.56 mmol), then the mixture was stirred at 25° C. for 12 hrs. LCMS showed the starting material was consumed completely and a main peak with desired mass was detected. The mixture was poured into water (20 mL) and stirred for 10 minutes, and filtered, then filter cake was collected and dried in vacuo to afford the titled compound (550 mg, crude) as a yellow solid, which was used for the next step directly. MS(M+H)⁺=295.1

### Step 5. Synthesis of (R)-4-((8-cyclohexyl-5, 7-dimethyl-6-oxo-5,6, 7, 8-tetrahydropteridin-2-yl)amino)-3-methoxybenzoic acid (7)

To the solution of 4-amino-3-methoxybenzoic acid (467.84 mg, 2.80 mmol) and (R)-2-chloro-8-cyclohexyl-5,7-dimethyl-7,8-dihydropteridin-6 (5H)-one (550 mg, 1.87 mmol) in EtOH (3.5 mL) and H₂O (12.5 mL) was added HCl (12 M, 342.06 μL) at 25° C., and then the mixture was stirred at 100° C. for 12 hrs. LCMS showed the starting material was consumed completely and a main peak with desired mass was detected. The mixture was concentrated to remove EtOH. The resulting mixture was diluted with H₂O (20 mL) and stirred for 10 minutes, then filtered, and filter cake was dried in vacuo to afford the titled compound (500 mg, 1.07 mmol, 57.31% yield, 91% purity) as a brown solid, which was used for the next step directly. MS(M+H)⁺=426.1

### Step 6. Synthesis of 4-(((R)-8-cyclohexyl-5, 7-dimethyl-6-oxo-5,6, 7, 8-tetrahydropteridin-2-yl)amino)-N-(2-(2-(2-((2-(2, 6-dioxopiperidin-3-yl)-1,3-dioxoisoindolin-4-yl)amino)ethoxy)ethoxy)ethyl)-3-methoxybenzamide (Compound 9)

To a solution of (R)-4-((8-cyclohexyl-5,7-dimethyl-6-oxo-5,6,7,8-tetrahydropteridin-2-yl)amino)-3-methoxybenzoic acid (70 mg, 164.52 μmol) in DMF (2 mL) were added HATU (93.83 mg, 246.78 μmol) and DIPEA (63.79 mg, 493.56 μmol, 85.97 μL), then the mixture was stirred at 30° C. for 10 minutes. Then 4-((2-(2-(2-aminoethoxy)ethoxy)ethyl)amino)-2-(2,6-dioxopiperidin-3-yl)isoindoline-1,3-dione (87.04 mg, 197.42 μmol, HCl) was added, then the mixture was stirred at 30° C. for 12 hrs. LCMS showed the starting material was consumed completely, and peak (60%) with desired mass was detected. The mixture was concentrated under reduced pressure, then purified by prep-HPLC (column: Waters Xbridge 150*25 mm*5 μm; mobile phase: [water (10 mM NH₄HCO₃)-ACN]; B %: 40%-70%, 10 min) to afford the titled compound (64.3 mg, 72.86 μmol, 44.29% yield, 92% purity) as a yellow solid. MS (M+H)⁺=812.2

¹H NMR (400 MHz, DMSO-d₆) δ=11.28-10.85 (m, 1H), 8.44 (d, J=8.4 Hz, 1H), 8.40 (br t, J=5.6 Hz, 1H), 7.88 (s, 1H), 7.64 (s, 1H), 7.54 (dd, J=7.3, 8.4 Hz, 1H), 7.51-7.45 (m, 2H), 7.09 (d, J=8.6 Hz, 1H), 7.01 (d, J=7.1 Hz, 1H), 6.58 (br t, J=5.6 Hz, 1H), 5.05 (dd, J=5.5, 12.8 Hz, 1H), 4.34 (q, J=6.6 Hz, 1H), 4.29-4.19 (m, 1H), 3.93 (s, 3H), 3.66-3.50 (m, 8H), 3.46-3.38 (m, 4H), 3.33-3.28 (m, 1H), 3.23 (s, 3H), 2.93-2.80 (m, 1H), 2.63-2.53 (m, 2H), 2.11-1.95 (m, 2H), 1.93-1.51 (m, 6H), 1.50-1.32 (m, 2H), 1.23 (d, J=6.6 Hz, 3H)

### Example 10: Synthesis of 4-(((R)-8-cyclopentyl-7-ethyl-5-methyl-6-oxo-5, 6,7, 8-tetrahydropteridin-2-yl)amino)-N-((1r,4R)-4-(4-(2-(2-((2-(2, 6-dioxopiperidin-3-yl)-1,3-dioxoisoindolin-4-yl)amino)ethoxy)ethyl) piperazin-1-yl)cyclohexyl)-3-methoxybenzamide (Compound 10)

### Synthetic Scheme:

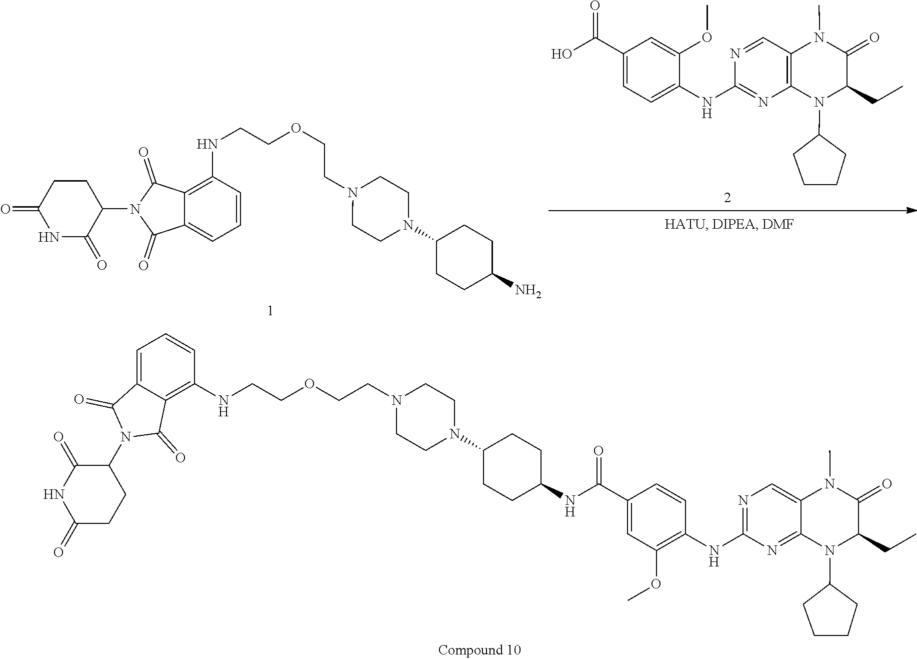 [SMILES: N[C@H]1CC[C@H](N2CCN(CCOCCNc3cccc4c3C(=O)N(C3CCC(=O)NC3=O)C4=O)CC2)CC1 ; CC[C@@H]1C(=O)N(C)c2cnc(Nc3ccc(C(=O)O)cc3OC)nc2N1C1CCCC1 ; CC[C@@H]1C(=O)N(C)c2cnc(Nc3ccc(C(=O)N[C@H]4CC[C@H](N5CCN(CCOCCNc6cccc7c6C(=O)N(C6CCC(=O)NC6=O)C7=O)CC5)CC4)cc3OC)nc2N1C1CCCC1]

### Experimental Procedure:

### Step 1. Synthesis of 4-(((R)-8-cyclopentyl-7-ethyl-5-methyl-6-oxo-5,6, 7, 8-tetrahydropteridin-2-yl)amino)-N-((1r,4R)-4-(4-(2-(2-((2-(2, 6-dioxopiperidin-3-yl)-1, 3-dioxoisoindolin-4-yl)amino)ethoxy)ethyl)piperazin-1-yl)cyclohexyl)-3-methoxybenzamide (Compound 10)

To a solution of (R)-4-((8-cyclopentyl-7-ethyl-5-methyl-6-oxo-5,6,7,8-tetrahydropteridin-2-yl)amino)-3-methoxybenzoic acid (80 mg, 188.02 μmol) in DMF (2 mL) were added HATU (78.64 mg, 206.83 μmol) and DIPEA (48.60 mg, 376.05 μmol, 65.50 μL), then the mixture was stirred at 20° C. for 10 minutes. Then to the mixture was added a solution of 4-((2-(2-(4-((1r,4r)-4-aminocyclohexyl)piperazin-1-yl)ethoxy)ethyl)amino)-2-(2,6-dioxopiperidin-3-yl)isoindoline-1,3-dione (158.81 mg, 282.03 μmol, HCl salt) and DIPEA (48.60 mg, 376.05 μmol, 65.50 μL) in DMF (2 mL) drop-wise at 20° C. and the resulting mixture was stirred at 20° C. for 1 hr. LCMS showed all starting materials were consumed completely and peak with desired mass was detected. The mixture was diluted with H₂O (8 mL), then extracted with EtOAc (8 mL×3). The combined organic layer was washed with brine (8 mL×3), then dried over Na₂SO₄, and then filtered. The filtrate was concentrated under reduced pressure. The residue was purified by prep-HPLC (column: Phenomenex Gemini-NX C₁₈ 75*30 mm*3 μm; mobile phase: [water (10 mM NH₄HCO₃)-ACN]; B %: 30%-60%, 8 min), then lyophilized to afford the titled compound (46.9 mg, 46.69 μmol, 24.83% yield, 93% purity) as a yellow solid. MS(M+H)⁺=934.2

¹H NMR (DMSO-d₆) δ: 11.02-11.18 (m, 1H), 8.41 (d, J=8.9 Hz, 1H), 8.02 (br d, J=7.7 Hz, 1H), 7.85 (s, 1H), 7.56-7.62 (m, 2H), 7.47 (dd, J=4.4, 2.6 Hz, 2H), 7.15 (d, J=8.6 Hz, 1H), 7.05 (d, J=7.0 Hz, 1H), 6.60 (t, J=5.6 Hz, 1H), 5.03-5.10 (m, 1H), 4.31-4.41 (m, 1H), 4.23 (dd, J=7.6, 3.5 Hz, 1H), 3.94 (s, 3H), 3.65-3.77 (m, 1H), 3.58-3.62 (m, 2H), 3.54 (br t, J=5.7 Hz, 2H), 3.44-3.50 (m, 2H), 3.25 (s, 3H), 2.84-2.98 (m, 1H), 2.60-2.64 (m, 1H), 2.54-2.59 (m, 2H), 2.52 (br d, J=2.0 Hz, 2H), 2.43-2.46 (m, 6H), 1.98-2.07 (m, 2H), 1.85-1.94 (m, 4H), 1.71-1.84 (m, 7H), 1.58-1.69 (m, 3H), 1.20-1.45 (m, 5H), 0.76 (t, J=7.4 Hz, 3H).

### Example 11: Synthesis of 4-(((R)-8-cyclopentyl-7-ethyl-5-methyl-6-oxo-5, 6,7, 8-tetrahydropteridin-2-yl)amino)-N-((1r,4R)-4-(4-(2-((2-(2, 6-dioxopiperidin-3-yl)-1, 3-dioxoisoindolin-4-yl)amino)ethyl) piperazin-1-yl)cyclohexyl)-3-methoxybenzamide (Compound 11)

### Synthetic Scheme:

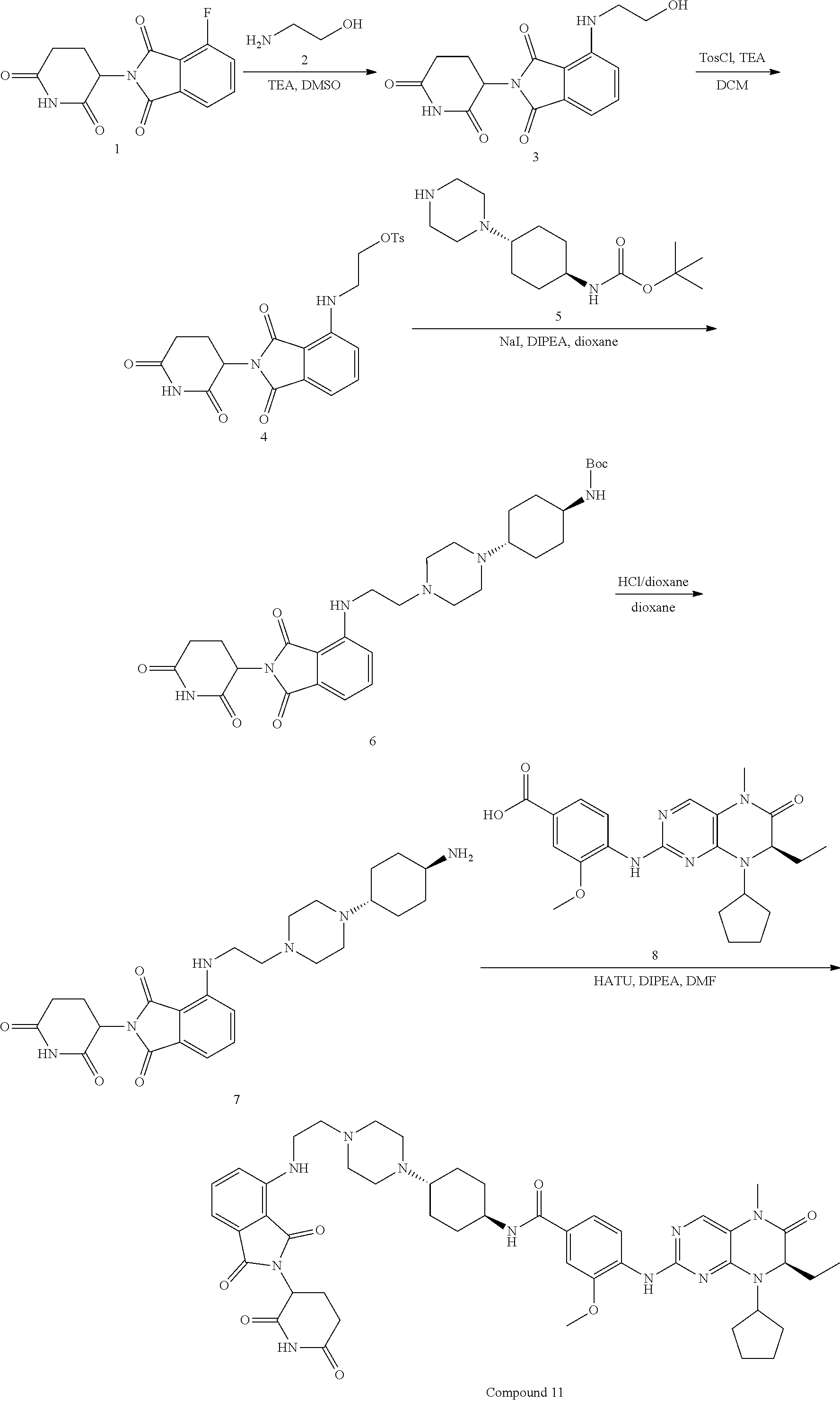

### Experimental Procedure:

### Step 1. Synthesis of 2-(2, 6-dioxopiperidin-3-yl)-4-((2-hydroxyethyl)amino)isoindoline-1,3-dione (3)

To a solution of 2-(2,6-dioxo-3-piperidyl)-4-fluoro-isoindoline-1,3-dione (5 g, 18.10 mmol) and 2-aminoethanol (1.33 g, 21.72 mmol, 1.31 mL) in DMSO (50 mL) was added TEA (5.50 g, 54.30 mmol, 7.56 mL) in one portion at 20° C., then the mixture was stirred at 80° C. for 16 hrs. LCMS showed starting material was consumed completely and peak with desired mass was detected. The mixture was diluted with H₂O (100 mL) and extracted with EtOAc (100 mL×5). The organic layer was washed with brine (100 mL×3), then dried over Na₂SO₄, and filtered to afford the titled compound (5.8 g, crude) as a green oil. MS(M+H)⁺=318.1

### Step 2. Synthesis of 2-((2-(2,6-dioxopiperidin-3-yl)-1,3-dioxoisoindolin-4-yl)amino)ethyl 4-methylbenzenesulfonate (4)

To a solution of 2-(2,6-dioxopiperidin-3-yl)-4-((2-hydroxyethyl)amino)isoindoline-1,3-dione (5.7 g, 17.96 mmol) in DCM (60 mL) were added TEA (5.45 g, 53.89 mmol, 7.50 mL) and TosCl (5.14 g, 26.95 mmol) in one portion at 20° C. and the resulting mixture was stirred at 20° C. for 16 hrs. LCMS showed starting material was consumed completely and peak with desired mass was detected. The mixture was concentrated under reduced pressure, then the residue was purified by flash silica gel chromatography (40 g SepaFlash® Silica Flash Column, Eluent of 0˜100% Ethyl acetate/Petroleum ether gradient @ 100 mL/min) to afford 4.1 g of product with 39% purity by LCMS. The product was purified by reversed-phase MPLC (method: FA; Column 330 g Flash Column Welch MLtimate XB_C₁₈ 20-40 μm; 120 A; Solvent for sample dissolution about 4.10 grams of sample dissolved in 50 mL of DMF; Flow rate 100 mL/min; Mobile phase MeCN/H₂O; Gradient B % 5-50% 25 min; 50% 20 min; Instrument TELEDYNE ISCO CombiFlashRf₁₅₀), and then lyophilized to afford the titled compound (320 mg, 665.14 μmol, 3.70% yield, 98% purity) as a yellow solid. MS (M+H)⁺=472.1

### Step 3. Synthesis of tert-butyl ((1r,4r)-4-(4-(2-((2-(2, 6-dioxopiperidin-3-yl)-1,3-dioxoisoindolin-4-yl)amino)ethyl)piperazin-1-yl)cyclohexyl)carbamate (6)

To a solution of 2-((2-(2,6-dioxopiperidin-3-yl)-1,3-dioxoisoindolin-4-yl)amino)ethyl 4-methylbenzenesulfonate (320 mg, 678.71 μmol) and tert-butyl ((1r,4r)-4-(piperazin-1-yl)cyclohexyl)carbamate (211.59 mg, 746.58 μmol) in dioxane (6 mL) were added NaI (20.35 mg, 135.74 μmol) and DIPEA (263.16 mg, 2.04 mmol, 354.66 μL) in one portion at 20° C., and then the mixture was stirred at 80° C. for 16 hrs. LCMS showed starting material was consumed completely and peak with desired mass was detected. The mixture was concentrated under reduced pressure, then purified by flash silica gel chromatography (10 g SepaFlash® Silica Flash Column, Eluent of 0˜100% Ethyl acetate/Petroleum ether to 0˜20% Dichloromethane/Methanol gradient @ 80 mL/min) to afford product A (556 mg) with 98% purity by LCMS. The product A was diluted with EtOAc (5 mL), then the organic layer was washed with H₂O (5 mL×3), then dried over Na₂SO₄, and filtrated and concentrated under reduced pressure to afford the titled compound (196 mg, 312.83 μmol, 46.09% yield, 93% purity) as a yellow solid. MS(M+H)⁺=583.3

### Step 4. Synthesis of 4-((2-(4-((1r,4r)-4-aminocyclohexyl)piperazin-1-yl)ethyl)amino)-2-(2, 6-dioxopiperidin-3-yl)isoindoline-1,3-dione (7)

To a solution of tert-butyl ((1r,4r)-4-(4-(2-((2-(2,6-dioxopiperidin-3-yl)-1,3-dioxoisoindolin-4-yl)amino)ethyl)piperazin-1-yl)cyclohexyl)carbamate (195 mg, 334.66 μmol) in dioxane (4 mL) was added HCl/dioxane (4 M, 8 mL) in one portion at 20° C., then the mixture was stirred for 1 hr. LCMS showed the starting material was consumed completely and peak with desired mass was detected. The mixture was concentrated under reduced pressure to afford the titled compound (190 mg, crude, HCl) as a yellow solid. MS(M+H)⁺=483.3

### Step 5. 4-(((R)-8-cyclopentyl-7-ethyl-5-methyl-6-oxo-5,6, 7, 8-tetrahydropteridin-2-yl)amino)-N-((1r,4R)-4-(4-(2-((2-(2, 6-dioxopiperidin-3-yl)-1,3-dioxoisoindolin-4-yl)amino)ethyl)piperazin-1-yl)cyclohexyl)-3-methoxybenzamide (Compound 11)

To a solution of (R)-4-((8-cyclopentyl-7-ethyl-5-methyl-6-oxo-5,6,7,8-tetrahydropteridin-2-yl)amino)-3-methoxybenzoic acid (80 mg, 188.02 μmol) in DMF (2 mL) were added HATU (78.64 mg, 206.83 μmol) and DIPEA (48.60 mg, 376.05 μmol, 65.50 μL), then the mixture was stirred at 20° C. for 10 minutes. Then to the mixture was added a solution of 4-((2-(4-((1r,4r)-4-aminocyclohexyl)piperazin-1-yl)ethyl)amino)-2-(2,6-dioxopiperidin-3-yl)isoindoline-1,3-dione (107.35 mg, 206.83 μmol, HCl) in DMF (2 mL) and DIPEA (48.60 mg, 376.05 μmol, 65.50 μL, 2 eq) drop-wise at 20° C., and the resulting mixture was stirred for 1 hr. LCMS showed the starting material was consumed completely and peak with desired mass was detected. The mixture was diluted with H₂O (10 mL), then extracted with EtOAc (10 mL×3). The organic layer was washed with brine (10 mL×3), then dried over Na₂SO₄, and filtered and concentrated. The residue was purified by prep-HPLC (column: Unisil 3-100 C₁₈ Ultra 150*50 mm*3 μm; mobile phase: [water (0.225% FA)-ACN]; B %: 10%-40%, 10 min), then lyophilized to afford 60.1 mg of product with 98% purity by HPLC. The product was re-purified by prep-HPLC (column: Waters Xbridge 150*25 mm*5 μm; mobile phase: [water (10 mM NH₄HCO₃)-ACN]; B %: 47%-77%, min), and then lyophilized to afford the titled compound (30.2 mg, 30.88 μmol, 16.42% yield, 91% purity) as a yellow solid. MS(M+H)⁺=890.4

¹H NMR (400 MHz, DMSO-d₆) δ=11.08 (br s, 1H), 8.44-8.36 (m, 1H), 8.03 (d, J=7.9 Hz, 1H), 7.84 (s, 1H), 7.63-7.55 (m, 2H), 7.51-7.43 (m, 2H), 7.09 (d, J=8.6 Hz, 1H), 7.03 (d, J=7.1 Hz, 1H), 6.73 (s, 1H), 5.06 (dd, J=5.4, 12.9 Hz, 1H), 4.35 (q, J=8.4 Hz, 1H), 4.23 (dd, J=3.4, 7.6 Hz, 1H), 3.93 (s, 3H), 3.77-3.68 (m, 1H), 3.24 (s, 3H), 2.93-2.83 (m, 1H), 2.59-2.53 (m, 6H), 2.46-2.35 (m, 4H), 2.30-2.15 (m, 2H), 2.05-1.98 (m, 2H), 1.94-1.71 (m, 11H), 1.68-1.57 (m, 3H), 1.50-1.17 (m, 6H), 0.76 (t, J=7.4 Hz, 3H)

### Experimental Example 1: Construction and Culture of HeLa LgBit (Plk1-HiBit KI) and HEK293T LgBit (BRD4-HiBit KI) Cell Lines

Cell lines were constructed by transfecting LgBit vector into HeLa and HEK293T cell lines to be stably expressed. Then, gRNA and a donor were created to express the HiBit amino acid sequence behind the C-terminus of the PLK1 and BRD4 proteins contained in each cell, and then inserted into the cells together with a vector capable of expressing CRISPR/Cas9. Only cells that had undergone knock-in after completion of insertion were selected and passaged to be used.

For cell counting, a cell counter (Thermo Fisher Scientific Inc., Catalog #AMQAX1000) and 0.4% trypan blue solution were used.

For cell culture, DMEM (Gibco, Cat. No. 11995-065; Lot. No. 2307627), FBS (Gibco, Cat. No. 16000-044; Lot. No. 2351176P), Penicillin/Streptomycin (Gibco, Cat. No. 15140-122; Lot. No. 2321150), 100 mm² cell culture dish (SPL, Cat. No. 20100), 150 mm² cell culture dish (SPL, Cat. No. 20150), 96-well white plate (SPL, Cat. No. 30196), PBS pH 7.4 (Gibco, Cat. No. 10010-023; Lot. No. 2085080), TrypLE™ Express (Gibco, Cat. No. 12605-010; Lot. No. 2070638), Counting Chamber (Hirschmann, Cat. No. 8100204), and 0.4% Trypan Blue Solution (DYNEBIO, Cat. No. CBT3710; Lot. No. 20190723) were used.

### Experimental Example 2: Treatment with Compounds of the Present Invention and Luciferase Assay

Compounds according to Comparative Examples and Examples were completely dissolved in DMSO (Sigma-Aldrich Cat. No. D2438, Lot. No. RNBJ6524) and used in the following experiments. The compound of Comparative Example was the HBL-4 compound disclosed in the document (see Mu et al., Biochem Biophys Res Commun., 2020, 521(4): 833), which targets PLK1 and BRD4 like the compounds of the present invention, and the DMSO-only treatment group was used as the control group.

As to HeLa LgBit (Plk1-HiBit KI), cells were released after thymidine block, and then treated with compounds, and the process was as follows. Thymidine (Sigma-Aldrich Cat. No. T9250-5G) was completely dissolved in DW and used in the experiment. For thymidine block, cells were treated with 2 mM thymidine and incubated for 24 hours. For release and chemical treatment, the medium was suctioned and washed with 1×PBS. TyppLE™ was added and the cells were incubated for 5 min in a 37° C. CO₂ incubator. Cells neutralized by adding complete medium were counted using the counter. For each well of a 96-well white plate (SPL), 3.3×10⁴ cells were seeded in a total culture medium volume of 150 μl and incubated in a CO₂ incubator.

As to HEK293T LgBit (BRD4-HiBit KI), 4×10⁴ cells were seeded in each well of a 96-well white plate (SPL) and cultured in a total culture medium volume of 150 μl.

Each cell line was incubated in a CO₂ incubator for 18 hours, and then Endurazine was added to each well to reach 4% of the total volume. Here, the concentration of each compound after the addition was 300 nM for HeLa LgBit (Plk1-HiBit KI) and 100 nM for HEK293T LgBit (BRD4-HiBit KI). Then, the wavelength of the plate reader was set to 470 to 480 nm, and luminescence was tracked in real time. Luminescence values at 9 hours for HeLa LgBit (Plk1-HiBit KI) and 8 hours for HEK293T LgBit (BRD4-HiBit KI) were calculated and displayed as a bar graph using an Excel program. Results thereof are shown in Table 2 and FIG. 1 below.

TABLE 2

| No. | PLK1 | BRD4 |
|---|---|---|
| Comparative Example 1 | 0.148 | 0.223 |
| Compound 4 | 0.208 | 0.692 |
| Compound 5 | 0.140 | 0.491 |
| Compound 6 | 0.313 | 0.731 |
| Compound 7 | 0.120 | 0.488 |
| Compound 8 | 0.204 | 0.394 |
| Compound 9 | 0.207 | 0.484 |
| Compound 10 | 0.343 | 0.629 |

As a result of the experiment, it was confirmed that Comparative Example, which is a known PROTAC, degraded both PLK1 and BRD4 at a level of 80% or more, however, the compounds of the present invention all degraded PLK1 at a level similar to Comparative Example, and degraded BRD4 at a level of about 30 to 60%, which showed relatively weak BRD4 degradation activity compared to Comparative Example. These results suggest that the compounds of the present invention simultaneously target PLK1 and BRD4 to be effective against cancer and neurological diseases, and have relatively small side effects that may occur due to excessive degradation of BRD4.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. The scope of the present disclosure is indicated by the following claims rather than the detailed description, and should be construed as including all changes or modifications derived from the meaning and scope of the claims and equivalent concepts within the scope of the present invention.

1. A compound represented by the following Formula I, a stereoisomer thereof or a pharmaceutically acceptable salt thereof: 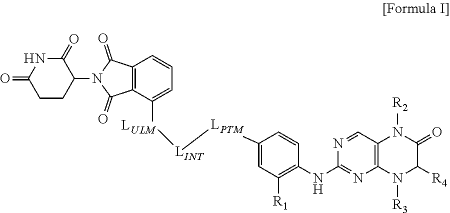
in Formula I above,
R₁ is —H, —CH₃ or —OCH₃,
R₂ is —H or —CH₃,
R₃ is -cyclopentyl or -cyclohexyl,
R₄ is —C₁₋₃alkyl,
L_{ULM} is —NH—CH₂CH₂— or 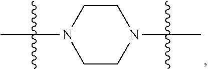 [SMILES: *N1CCN(*)CC1]
L_{INT} is —(OCH₂CH₂)ₙ—, —(C═O)—CH₂CH₂—(C═O)—, —CH₂CH₂—(C═O)—, —CH₂—(C═O)—, 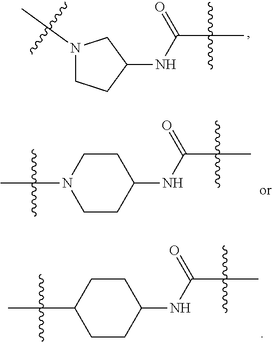 [SMILES: *C(=O)NC1CCN(*)C1 ; *C(=O)NC1CCN(*)CC1 ; *C(=O)NC1CCC(*)CC1]
(wherein n is an integer of 2 to 4), and
L_{PTM} is —NH—(C═O)—, or

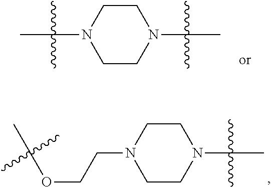 [SMILES: *N1CCN(*)CC1 ; *OCCN1CCN(*)CC1]

2. The compound, the stereoisomer thereof or the pharmaceutically acceptable salt thereof according to claim 1, wherein the compound represented by the Formula I is selected from Compounds 1 to 11:

| Com- | |
|---|---|
| pound | Structure |
| 1 | 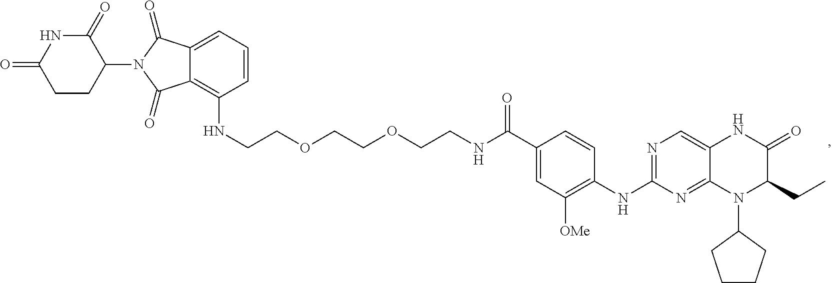 [SMILES: CC[C@@H]1C(=O)Nc2cnc(Nc3ccc(C(=O)NCCOCCOCCNc4cccc5c4C(=O)N(C4CCC(=O)NC4=O)C5=O)cc3OC)nc2N1C1CCCC1] |
| 2 | 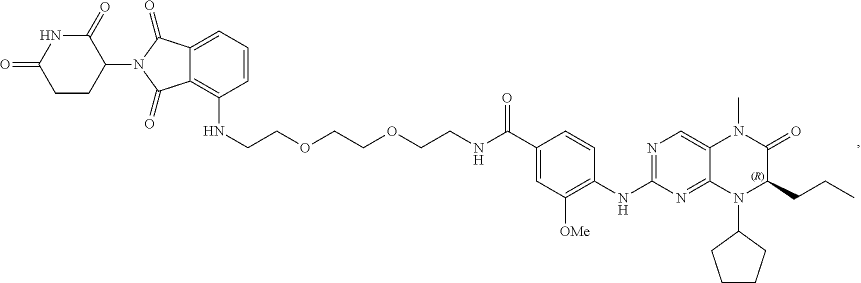 |
| 3 | 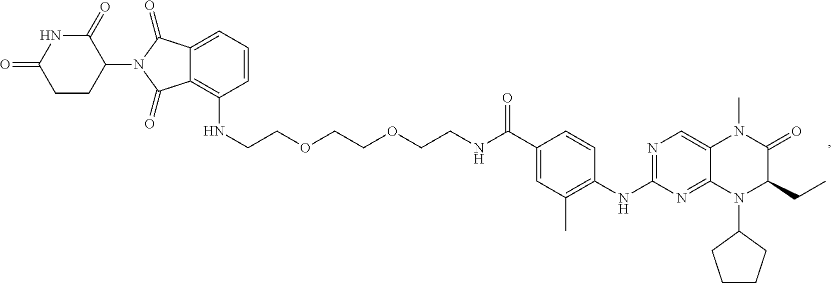 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3C)nc2N1C1CCCC1] |
| 4 | 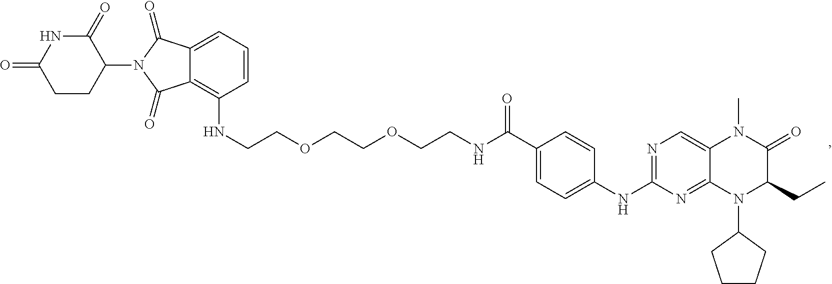 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3)nc2N1C1CCCC1] |
| 5 | 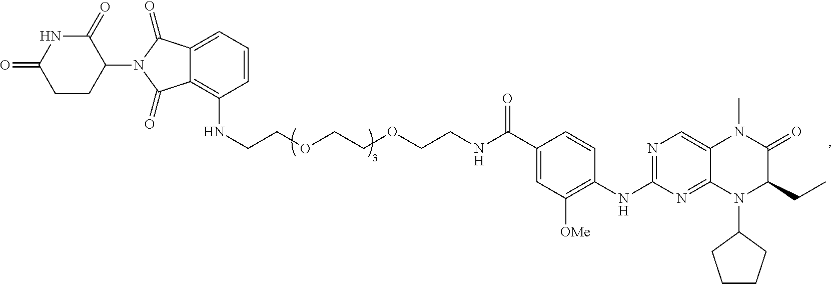 |
| 6 | 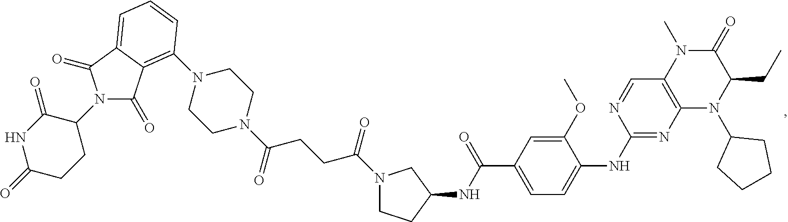 [SMILES: CC[C@@H]1C(=O)N(C)c2cnc(Nc3ccc(C(=O)N[C@H]4CCN(C(=O)CCC(=O)N5CCN(c6cccc7c6C(=O)N(C6CCC(=O)NC6=O)C7=O)CC5)C4)cc3OC)nc2N1C1CCCC1] |
| 7 | 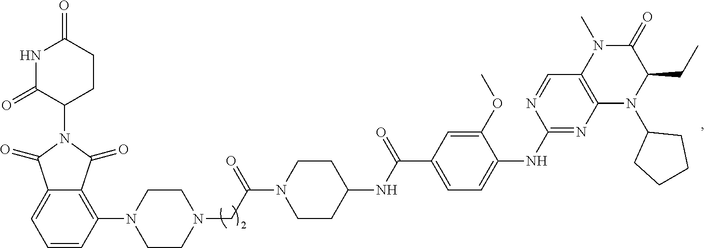 |
| 8 | 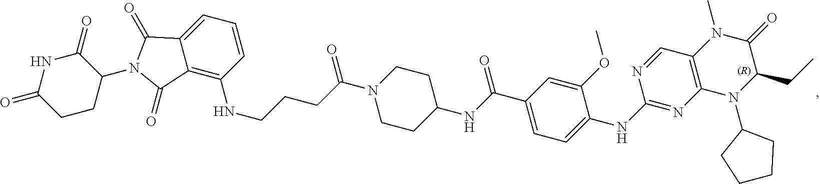 |
| 9 | 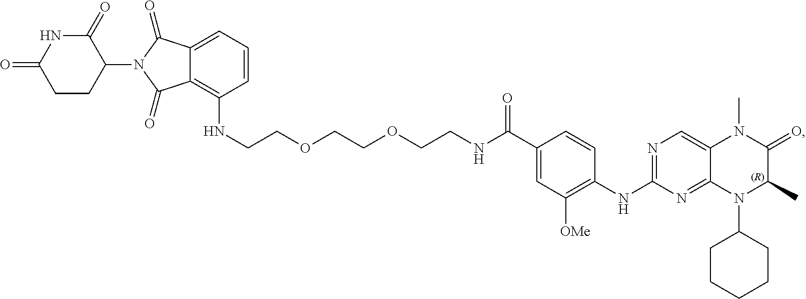 |
| 10 | 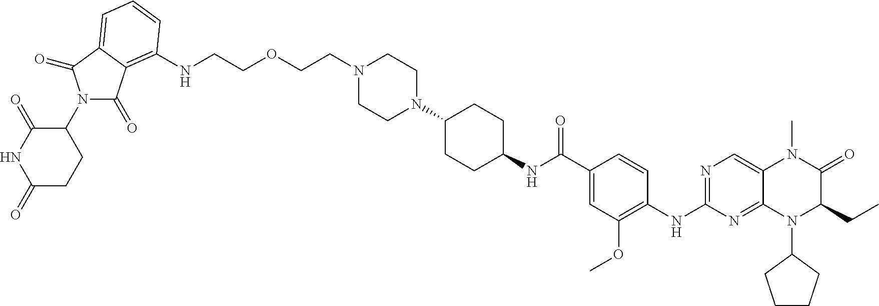 [SMILES: CC[C@@H]1C(=O)N(C)c2cnc(Nc3ccc(C(=O)N[C@H]4CC[C@H](N5CCN(CCOCCNc6cccc7c6C(=O)N(C6CCC(=O)NC6=O)C7=O)CC5)CC4)cc3OC)nc2N1C1CCCC1] |
| 11 | 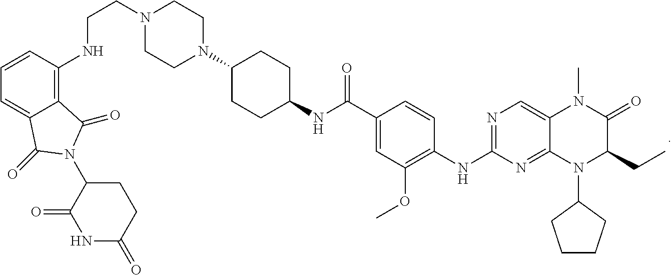 [SMILES: CC[C@@H]1C(=O)N(C)c2cnc(Nc3ccc(C(=O)N[C@H]4CC[C@H](N5CCN(CCNc6cccc7c6C(=O)N(C6CCC(=O)NC6=O)C7=O)CC5)CC4)cc3OC)nc2N1C1CCCC1] |

3. A pharmaceutical composition, comprising the compound of claim 1, the stereoisomer thereof or the pharmaceutically acceptable salt thereof as an active ingredient.

4. A method for preventing or treating cancer or neurological diseases, comprising administering the compound of claim 1, the stereoisomer thereof or the pharmaceutically acceptable salt thereof to a subject in need thereof.
